(19) United States
(12) Patent Application Publication
(10) Pub. No.: US 2025/0345179 A1
(43) Pub. Date: Nov. 13, 2025

(54) BONE IMPLANTS, SYSTEMS, AND METHODS

(71) Applicant: CATALYST ORTHOSCIENCE INC., Naples, FL (US)

(72) Inventors: Matthew BUDGE, Tualatin, OR (US); Edward Scott PAXTON, Barrington, RI (US); Ben SZERLIP, Dublin, OH (US); John BROWN, Peoria, AZ (US); Dinesh KOKA, Winter Park, FL (US); Ephraim AKYUZ, Salt Lake City, UT (US)

(21) Appl. No.: 18/860,646

(22) PCT Filed: Apr. 27, 2023
(86) PCT No.: PCT/US2023/020272

§ 371 Date: Oct. 26, 2024

Related U.S. Application Data
(60) Provisional application No. 63/335,253, filed on Apr. 27, 2022.

(51) Int. Cl. A61F 2/40; A61F 2/30

(52) U.S. Cl. CPC A61F 2/4014; A61F 2002/30112; A61F 2002/30159; A61F 2002/30578; A61F 2002/4022; A61F 2002/4033

(57) ABSTRACT

A compression fastener may include a shaft and a helical thread disposed about the shaft. The shaft may include a proximal end, a distal end, a proximal shaft portion, and a distal shaft portion. The helical thread may include at least one concave undercut surface and a plurality of pitches that may include at least one first pitch along the proximal shaft portion and at least one second pitch along the distal shaft portion. The at least one concave undercut surface may be angled towards one of the proximal end and the distal end of the shaft, and the at least one first pitch and the at least one second pitch may not be equal to each other.

### TECHNICAL FIELD

The present disclosure relates to bone implants, systems, and methods. More specifically, the present disclosure relates to bone implants, systems, and methods with improved structures and techniques for aligning, attaching, and maintaining one or more bone fragments in their proper anatomic orientations.

### BACKGROUND

Joint arthroplasty procedures are conducted to restore the function of an unhealthy joint. Typically, these procedures involve replacing the unhealthy natural articular surfaces of the joint with artificial articular surfaces. The new artificial articular surfaces are typically anchored into the adjacent bones to maintain long term stability. For example, in shoulder arthroplasty procedures, a humeral implant may be attached to the humerus and a glenoid implant may be attached to the glenoid or scapula.

In certain shoulder joint injuries, the proximal portion of the humerus may also be fractured. In these instances, anatomic and/or reverse shoulder arthroplasty techniques may be utilized in an attempt to re-align and re-attach the broken bone fragments back in their original anatomic locations, in addition to replacing one or more articular surfaces of the shoulder joint.

However, maintaining proper re-alignment and re-attachment of the broken bone fragments in their original anatomic locations during the healing process has proven difficult in view of the many rotational forces that are placed on these bone fragments by the muscles surrounding the shoulder joint during the healing process.

Accordingly, bone implants, systems, and methods with improved structures and techniques for re-aligning, re-attaching, and maintaining proper anatomic orientations for one or more bone fragments would be desirable.

### SUMMARY

The various bone implants, systems, and methods of the present disclosure have been developed in response to the present state of the art, and in particular, in response to the problems and needs in the art that have not yet been fully solved by currently available bone implants, systems, and methods. In some embodiments, the bone implants, systems, and methods of the present disclosure may provide improved fixation for bone fragments that experience rotational forces.

In some embodiments, a bone implant may include a rim component at a proximal end of the bone implant, a stem attachment feature at a distal end of the bone implant, a body portion intermediate the rim component and the stem attachment feature, and a raised ridge structure coupled to and projecting away from the exterior surface of the body portion. The body portion may include an exterior surface having a posterior-facing surface, a lateral-facing surface, and an anterior-facing surface. The raised ridge structure may include: a posterior ridge portion projecting away from the posterior-facing surface of the body portion between the proximal end and the distal end of the bone implant; an anterior ridge portion projecting away from the anterior-facing surface of the body portion between the proximal end and the distal end of the bone implant; and a superior ridge portion projecting away from the lateral-facing surface of the body portion adjacent the rim component and intermediate the posterior ridge portion and the anterior ridge portion. In some embodiments, the superior ridge portion may be continuously joined with at least one of the posterior ridge portion and the anterior ridge portion.

In some embodiments, a posterior end of the superior ridge portion may couple with a proximal end of the posterior ridge portion to continuously join the posterior ridge portion with the superior ridge portion, and an anterior end of the superior ridge portion may couple with a proximal end of the anterior ridge portion to continuously join the anterior ridge portion with the superior ridge portion.

In some embodiments, the raised ridge structure may also include a first curved portion intermediate the posterior ridge portion and the superior ridge portion, and a second curved portion intermediate the superior ridge portion and the anterior ridge portion. The first curved portion may couple intermediate the anterior end of the superior ridge portion and the proximal end of the posterior ridge portion to continuously join the superior ridge portion with the posterior ridge portion, and the second curved portion may couple intermediate the anterior end of the superior ridge portion and the proximal end of the anterior ridge portion to continuously join the superior ridge portion with the anterior ridge portion.

In some embodiments, the raised ridge structure may have a C-shape when viewed from a lateral direction.

In some embodiments, the raised ridge structure may include a ridge top having a rounded convex surface that defines an apex line extending along the ridge top.

In some embodiments, the apex line may be a continuous apex line that extends along the ridge top.

In some embodiments, the apex line may be a discrete apex line that extends along a discrete ridge top portion.

In some embodiments, a bone implant may include a body portion having a proximal end, a distal end, and an exterior surface extending between the proximal end and the distal end of the body portion. The exterior surface may include a posterior-facing surface, a lateral-facing surface, and an anterior-facing surface. The bone implant may also include a raised ridge structure coupled to and projecting away from the exterior surface of the body portion. The raised ridge structure may include a posterior ridge portion projecting away from the posterior-facing surface of the body portion between the proximal end and the distal end of the body portion, and an anterior ridge portion projecting away from the anterior-facing surface of the body portion between the proximal end and the distal end of the bone implant. The bone implant may also include a first bone fragment basin configured to receive a first bone fragment therein, the first bone fragment basin defined by the lateral-facing surface enclosed between the posterior ridge portion and the anterior ridge portion, as well as a second bone fragment basin configured to receive a second bone fragment therein, the second bone fragment basin defined by the anterior-facing surface enclosed between the anterior ridge portion and a rim component of the bone implant. A first surface of the posterior ridge portion defining a first side of the first bone fragment basin may be shaped to abut a first surface of the first bone fragment and prevent posterior rotational movement of the first bone fragment relative to the bone implant. A first surface of the anterior ridge portion defining a second side of the first bone fragment basin may be shaped to abut a second surface of the first bone fragment and prevent anterior rotational movement of the first bone fragment relative to the bone implant. A second surface of the anterior ridge portion defining the second bone fragment basin may be shaped to abut a first surface of the second bone fragment and prevent posterior rotational movement of the second bone fragment relative to the bone implant. A first surface of the rim component defining the second bone fragment basin may be shaped to abut a second surface of the second bone fragment and prevent anterior rotational movement of the second bone fragment relative to the bone implant.

In some embodiments, the raised ridge structure may also include a superior ridge portion projecting away from the lateral-facing surface of the body portion adjacent the rim component and intermediate the posterior ridge portion and the anterior ridge portion.

In some embodiments, a first surface of the superior ridge portion defining a third side of the first bone fragment basin may be shaped to abut a third surface of the first bone fragment and prevent superior rotational movement of the first bone fragment relative to the bone implant.

In some embodiments, at least one of: the first surface of the posterior ridge portion, the first surface of the anterior ridge portion, and the first surface of the superior ridge portion comprises a concave surface may comprise a concave surface.

In some embodiments, the superior ridge portion may be continuously joined with at least one of the posterior ridge portion and the anterior ridge portion.

In some embodiments, the first surface of the rim component may be angled toward a proximal end of the anterior ridge portion.

In some embodiments, the first surface of the rim component may be configured to abut the second surface of the second bone fragment to prevent anterior rotational movement and superior rotational movement of the second bone fragment relative to the bone implant.

In some embodiments, a bone implant may include a body portion and a raised ridge structure. The body portion may include a longitudinal axis, a proximal end, a distal end, and an exterior surface extending between the proximal end and the distal end of the body portion. The exterior surface may include a posterior-facing surface, a lateral-facing surface, and an anterior-facing surface. The raised ridge structure may be coupled to and projecting away from the exterior surface of the body portion. The raised ridge structure may include a posterior ridge portion projecting away from the posterior-facing surface of the body portion between the proximal end and the distal end of the body portion, and an anterior ridge portion projecting away from the anterior-facing surface of the body portion between the proximal end and the distal end of the bone implant. A first apex of the posterior ridge portion may project away from the longitudinal axis of the body portion along a posterior direction. A second apex of the anterior ridge portion may project away from the longitudinal axis of the body portion along an antero-lateral direction. An angle formed between the first apex and the second apex, relative to the longitudinal axis of the body portion, may be greater than ninety degrees and less than one hundred and forty five degrees.

In some embodiments, the posterior ridge portion may include a proximal region, a distal region, and a middle region intermediate the proximal region and the distal region. The proximal region of the posterior ridge portion may curve toward the proximal end of the body portion along a proximal direction, the distal region of the posterior ridge portion may curve toward the distal end of the body portion along a distal direction, and the middle region of the posterior ridge portion projects away from the body portion posteriorly relative to the proximal region and the distal region of the posterior ridge portion.

In some embodiments, the posterior ridge portion may comprise a C-shape when viewed laterally.

In some embodiments, the raised ridge structure may include a superior ridge portion that is continuously joined with at least one of the posterior ridge portion and the anterior ridge portion.

In some embodiments, the body portion may include a maximum lateral transverse width, the raised ridge structure may include a maximum transverse first basin width intermediate the posterior ridge portion and the anterior ridge portion, and the maximum transverse first basin width may not be less than 70% of the maximum lateral transverse width.

In some embodiments, the body portion may include a first basin having a first plain surface bounded between a lateral plane and a medial plane that are placed adjacent the first plain surface, such that: the lateral plane and the medial plane may be parallel to each other; at least one maximum surface point on the first plain surface may be contained within the lateral plane; at least one minimum surface point on the first plain surface may be contained within the medial plane; and a departure distance between the lateral plane and the medial plane may be less than 10% of a maximum length of the first plain surface.

These and other features and advantages of the present disclosure will become more fully apparent from the following description and appended claims or may be learned by the practice of the bone implants, systems, and methods set forth hereinafter.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings in which:

FIG. 1 illustrates an exploded view of a left humeral implant system, according to an embodiment of the present disclosure;

FIG. 2 illustrates a side view of the left humeral implant system of FIG. 1, after assembly;

FIG. 3 illustrates a rear perspective view of a right humeral implant system, according to an embodiment of the present disclosure;

FIG. 4 illustrates a rear perspective view of the left humeral implant system shown in FIG. 2;

FIG. 5 illustrates an exploded view of a left humeral implant system, according to another embodiment of the present disclosure;

FIG. 6 illustrates a side view of the left humeral implant system of FIG. 5, after assembly;

FIG. 7 illustrates a front perspective view of the left humeral implant system shown in FIG. 6;

FIG. 8 illustrates an exploded view of a right humeral implant system, according to another embodiment of the present disclosure;

FIG. 9 illustrates a side view of the right humeral implant system of FIG. 8, after assembly;

FIG. 10 illustrates a cross-sectional side view of the right humeral implant system shown in FIG. 9;

FIG. 11A illustrates an antero-lateral perspective view of a right humeral implant, according to an embodiment of the present disclosure;

FIG. 11B illustrates a postero-lateral perspective view of the right humeral implant of FIG. 11A;

FIG. 12A illustrates an antero-lateral perspective view of a left humeral implant, according to an embodiment of the present disclosure;

FIG. 12B illustrates a postero-lateral perspective view of the left humeral implant of FIG. 12A;

FIG. 12C illustrates an antero-medial perspective view of the left humeral implant of FIG. 12A;

FIG. 12D illustrates a postero-medial perspective view of the left humeral implant of FIG. 12A;

FIG. 12E illustrates a medial top perspective view of the left humeral implant of FIG. 12A;

FIG. 12F illustrates a lateral bottom perspective view of the left humeral implant of FIG. 12A;

FIG. 12G illustrates a medial bottom view of the left humeral implant of FIG. 12A;

FIG. 12H illustrates a lateral top view of the left humeral implant of FIG. 12A;

FIG. 12I illustrates a posterior side view of the left humeral implant of FIG. 12A;

FIG. 12J illustrates an anterior side view of the left humeral implant of FIG. 12A;

FIG. 12K illustrates a top perspective view of the left humeral implant of FIG. 12A;

FIG. 12L illustrates a cross-sectional view of the left humeral implant shown in FIG. 12K;

FIG. 12M illustrates a lateral side view of the left humeral implant of FIG. 12A;

FIG. 12N illustrates an anterior side view of the left humeral implant of FIG. 12A;

FIG. 12O illustrates an antero-lateral perspective view of the left humeral implant of FIG. 12A;

FIG. 12P illustrates a cross-sectional view of the left humeral implant shown in FIG. 120;

FIG. 13A illustrates an anterior view of a right humeral implant, according to another embodiment of the present disclosure;

FIG. 13B illustrates an antero-lateral view of the right humeral implant of FIG. 13A;

FIG. 13C illustrates a lateral view of the right humeral implant of FIG. 13A;

FIG. 13D illustrates a postero-lateral view of the right humeral implant of FIG. 13A;

FIG. 14A illustrates an anterior view of a right humeral implant, according to another embodiment of the present disclosure;

FIG. 14B illustrates an antero-lateral view of the right humeral implant of FIG. 14A;

FIG. 14C illustrates a lateral view of the right humeral implant of FIG. 14A;

FIG. 14D illustrates a postero-lateral view of the right humeral implant of FIG. 14A;

FIG. 15 illustrates an antero-lateral perspective view of a left humeral implant system relative to a humerus, a greater tuberosity bone fragment, and a lesser tuberosity bone fragment before implantation;

FIG. 16 illustrates an antero-lateral perspective view of the left humeral implant system of FIG. 15, after implantation;

FIG. 17 illustrates a close-up anterior view of the installed implant from FIG. 15;

FIG. 18 illustrates a close-up lateral view of the installed implant from FIG. 15;

FIG. 19 illustrates a close-up postero-lateral view of the installed implant from FIG. 15;

FIG. 20A illustrates an implant system, according to another embodiment of the present disclosure;

FIG. 20B illustrates the implant system of FIG. 20A with a bone fragment secured thereto;

FIG. 21A illustrates a lateral side view of a humeral implant, according to another embodiment of the present disclosure;

FIG. 21B illustrates a cross-sectional side view of the humeral implant of FIG. 21A;

FIG. 21C illustrates a partial top view of the humeral implant of FIG. 21A;

FIG. 22 illustrates a cross-sectional view of a humeral implant, according to an embodiment of the present disclosure;

FIG. 23 illustrates a lateral view of a humeral implant, according to an embodiment of the present disclosure;

FIG. 24 illustrates an anterior view of a right humeral implant, according to an embodiment of the present disclosure;

FIG. 25 illustrates methods for installing augments and bone grafts, according to embodiments of the present disclosure;

FIG. 26 illustrates a side view of a humeral implant system, according to embodiments of the present disclosure;

FIG. 27 illustrates a top view of a humeral implant system, according to embodiments of the present disclosure;

FIG. 28 illustrates a method of securing sutures to suture holes, according to an embodiment of the present disclosure; and

FIG. 29 illustrates a method of securing sutures to suture holes, according to another embodiment of the present disclosure.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the implants, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

Although the following detailed description utilizes the humerus as an example application for the concepts that are disclosed herein, it will also be understood that the general concepts, structures, systems, and techniques that are disclosed or contemplated herein may be adapted for use in any bone or joint of the body.

As used herein, the terms bone implant, long bone implant, metaphysis implant, arthroplasty implant, humeral implant, etc., can comprise any implant (having any structure or shape) that is implantable within bone that may (or may not) utilize any raised ridge structure morphology described or contemplated herein, including, but not limited to: shoulder bone implants, knee bone implants, ankle bone implants, hand/wrist/foot/finger/toe bone implants, arm/elbow implants, leg/hip bone implants, spinal bone implants, etc.

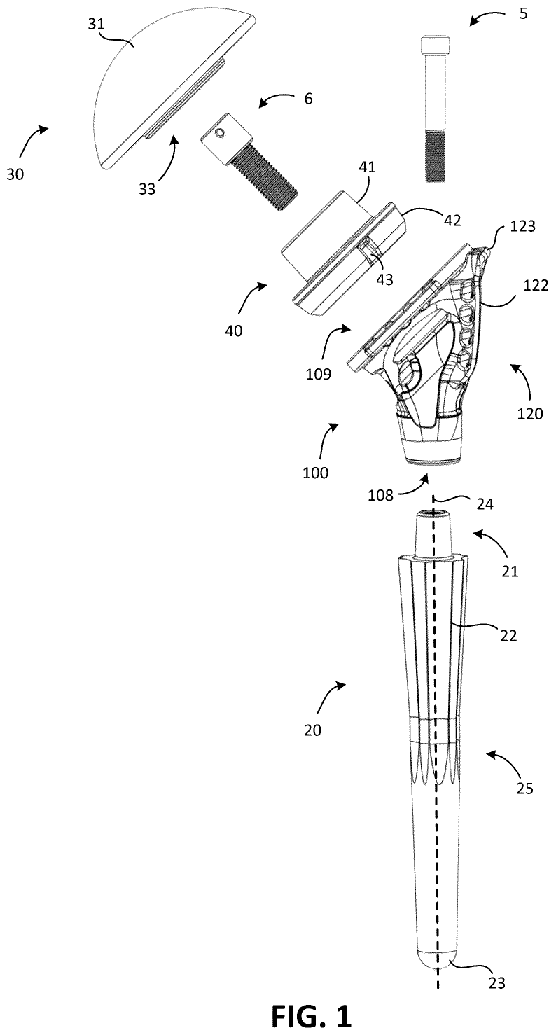

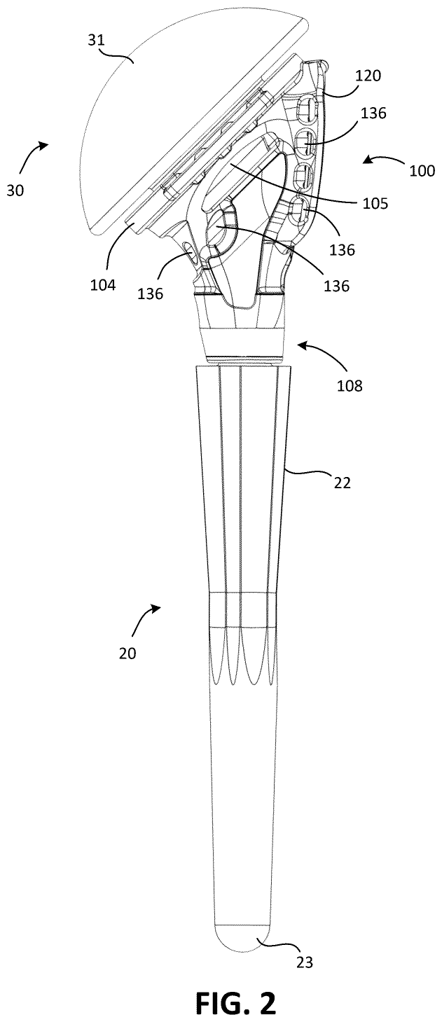

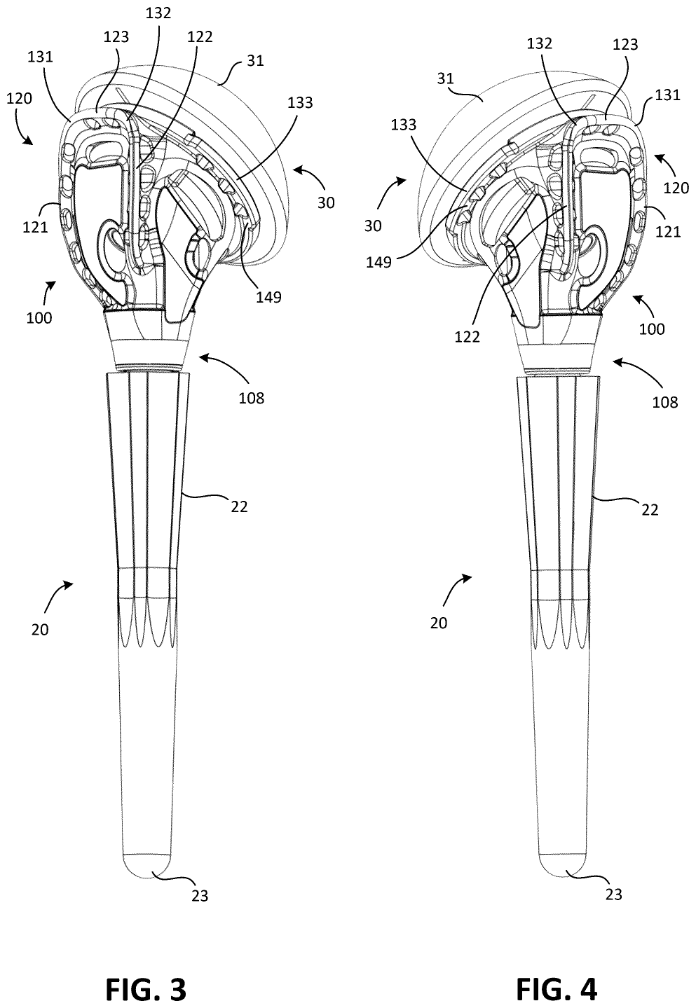

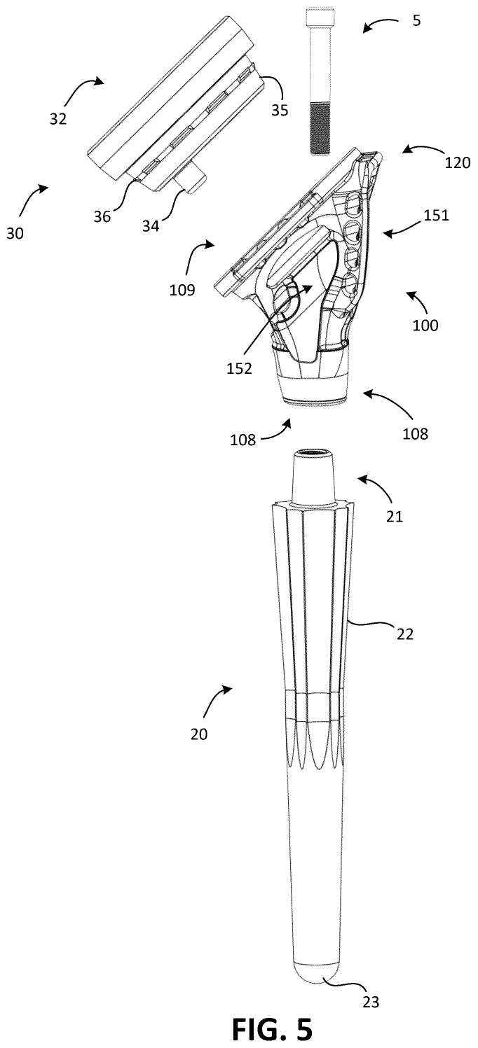

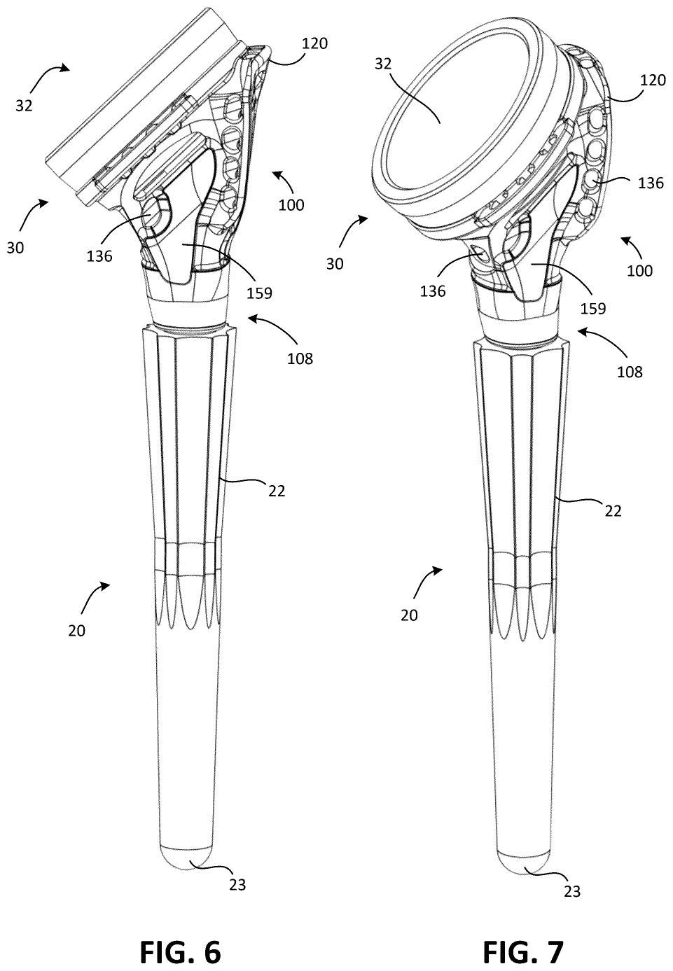

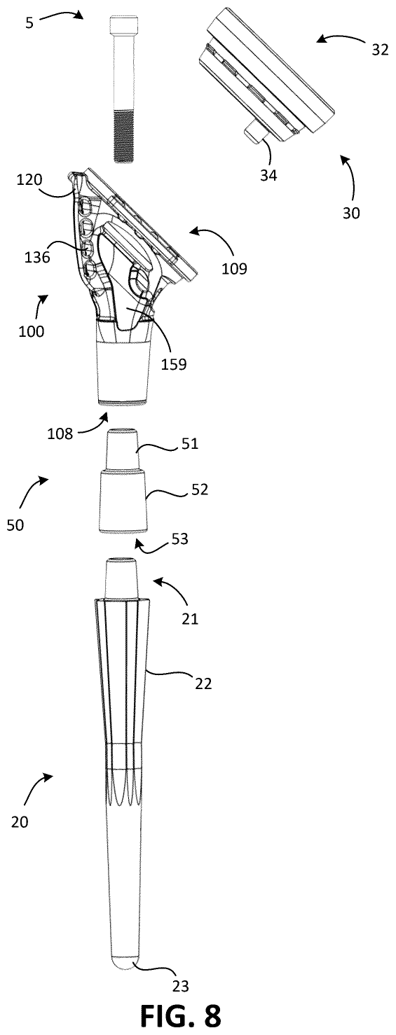

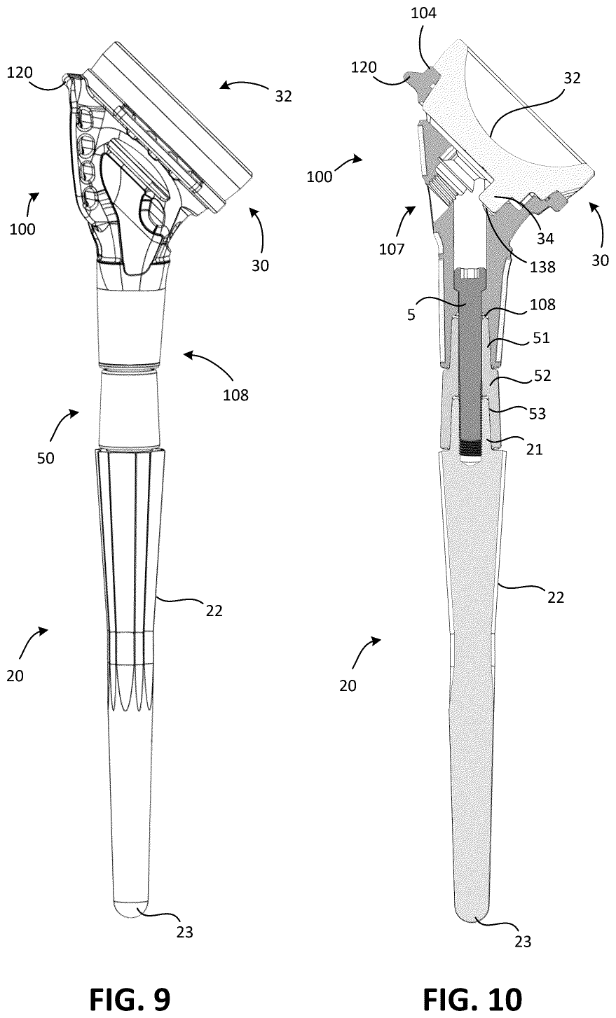

FIGS. 1-10 illustrate various views of different example humeral implant systems, according to embodiments of the present disclosure. Specifically: FIG. 1 shows an anatomic left humeral implant system; FIG. 2 shows the FIG. 1 implant system assembled together; FIGS. 3 and 4 show anatomic right and left humeral implant systems (e.g., mirror images of each other); FIG. 5 shows a reverse left humeral implant system; FIGS. 6 and 7 show the FIG. 5 implant system assembled together; FIG. 8 shows a reverse right humeral implant system; FIG. 9 shows the FIG. 8 implant system assembled together; and FIG. 10 shows a cross-sectional view of the FIG. 9 implant system. However, it will also be understood that any number of different humeral implant systems may be created by mixing and matching any of the implant components, structures, or features disclosed or contemplated herein in any number of different combinations.

In general, the implant systems shown in FIGS. 1-10 may comprise various components including, but not limited to: a bone implant 100 couplable with a stem 20 (e.g., via a stem fastener 5), an articular member 30, an adapter 40 (e.g., couplable to the bone implant 100 via an adapter fastener 6), and a spacer 50. In these embodiments, the implant systems may comprise various modular components. However, it will also be understood that any of the implant systems disclosed or contemplated herein may comprise one or more integrated components (e.g., in some embodiments the bone implant 100 and stem 20 may be formed as a single integral component, etc.).

The bone implant 100 may also be referred to herein as a long bone implant, a metaphysis implant, an arthroplasty implant, a humeral implant, etc.

In some embodiments, the stem 20 may include a stem body 25 having a central axis 24 that may be straight, curved, or combinations thereof.

In some embodiments, a transverse cross-sectional width of the stem body 25 may generally taper in width moving proximally to distally along the stem body 25.

In some embodiments, the stem 20 may include more than one tapered section along its length, or no tapered sections along its length.

In some embodiments, a distal end of the stem 20 may comprise a rounded distal end 23. However, the distal end of the stem 20 may comprise other shape.

In some embodiments, the stem 20 may include one or more ribs 22, extensions, fins, arms, pillars, rails, bars, cylinders, columns, etc., or any other geometrical shapes along all or part of its length. The one or more ribs 22 may be configured to provide additional fixation and/or rotational stability for the bone implant 100.

In some embodiments, the one or more ribs 22 may include sharp points, curved ends/radii, flat edges/rims, etc., or combinations thereof.

In some embodiments, the one or more ribs 22 may be radially arranged with symmetric spacing (or non-symmetric/random/alternating spacing) about the stem body 25.

In some embodiments, the one or more ribs 22 may vary in geometry and/or width along the stem body 25. For example, the one or more ribs 22 may have widths that taper at different rates along the stem body 25.

In some embodiments, a proximal end of the stem 20 may include a connection feature, such as a stem post 21, configured to couple with the bone implant 100.

In some embodiments, the stem post 21 may comprise a taper lock or Morse taper shape to secure the stem post 21 to the bone implant 100.

In some embodiments, the stem post 21 may be internally threaded to interface with the stem fastener 5 to supplement the taper lock connection of the stem post 21.

In some embodiments, the stem 20 may include any surface coating that may be useful to enhance mechanical fixation, provide bone integration potential, create anti-bacterial surfaces, etc. The surface coating may include a grit blasting, sintered beads, titanium or calcium phosphate porous coatings, additively manufactured porous structures, biofilms, etc., or any combinations thereof.

In some embodiments, the bone implant 100 may have an interior socket 109 shaped to receive an articular member 30 therein.

In some embodiments, the interior socket 109 of the bone implant 100 may also include one or more cutouts 105 and an inserter interface feature 107 that may be shaped, threaded, or otherwise configured to interface with one or more inserter and/or removal instruments (not shown) to facilitate insertion and/or removal of the bone implant 100 from bone.

In some embodiments, the articular member 30 may comprise an anatomical articular member that comprises a convex articular surface 31.

In some embodiments, the articular member 30 may comprise a reverse articular member that comprises a concave articular surface 32.

In some embodiments, the articular member 30 may comprise an articular member recess 33 formed therein that is configured to receive an adapter post 41 that projects from an adapter base 42 of the adapter 40 (e.g., see FIG. 1).

In some embodiments, the adapter post 41 may include a taper lock or Morse taper shape to secure the articular member 30 to the adapter 40.

In some embodiments, the adapter 40 may include an adapter recess 43 formed therein and configured to receive a tab 106 projecting from the bone implant 100 to prevent rotational movement of the adapter 40 within the interior socket 109 of the bone implant 100.

In some embodiments, the articular member 30 may include an articular member base 35 having an articular member post 34 projecting therefrom and configured to be received within an articular member post recess 138 formed in the bone implant 100.

In some embodiments, the articular member 30 may also include one or more snap-fit features 36 that may be formed about the articular member base 35 and configured to secure the articular member 30 within the interior socket 109 of the bone implant 100.

In some embodiments, the spacer 50 may include a spacer post 51 having a taper lock or Morse taper shape that is receivable within a stem attachment feature 108 of the bone implant 100, as shown in FIG. 10. The stem attachment feature 108 of the bone implant 100 may also include a taper lock or Morse taper shape. In this manner, the spacer 50 may provide additional height for the implant system.

In some embodiments, the spacer 50 may also include a spacer base 52 having a spacer recess 53 formed therein with a taper lock or Morse taper shape that is configured to receive the stem post 21.

In some embodiments, the bone implant 100, stem 20, and/or spacer 50 may be further securable to each other via the stem fastener 5 (see FIG. 10).

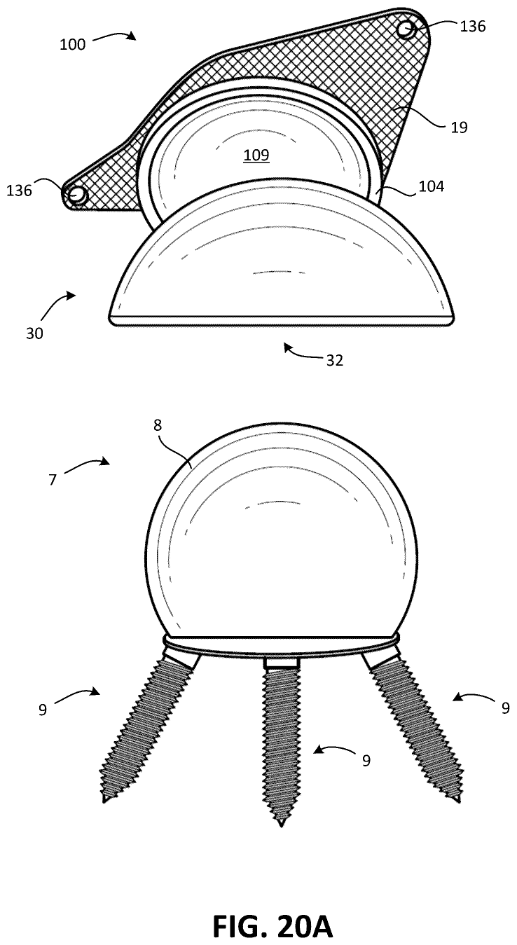

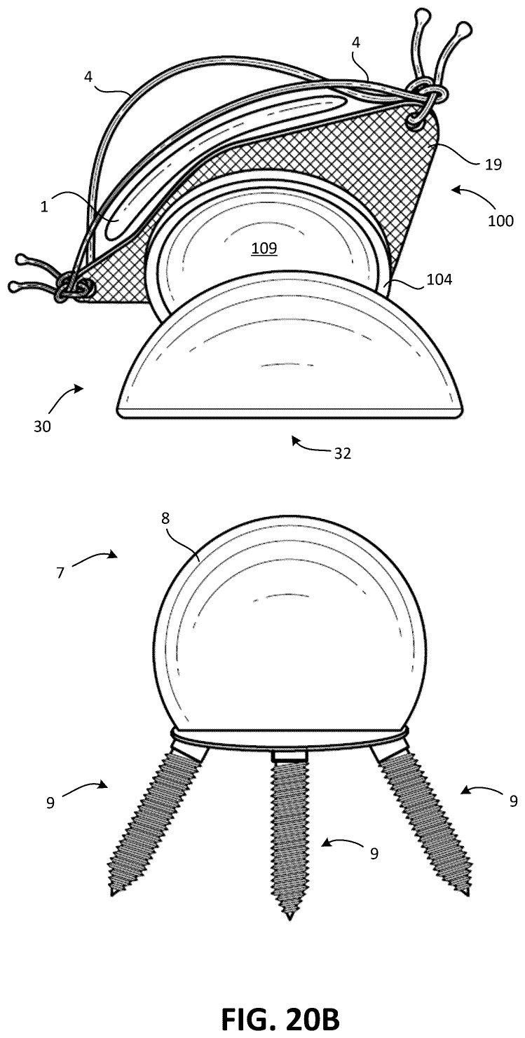

FIGS. 20A, 20B, 22, and 25 illustrate various views of additional example humeral implant systems, according to embodiments of the present disclosure. These humeral implant systems comprise reverse humeral implant systems including a glenosphere 7 with a convex glenosphere articular surface 8 that may be couplable to a glenoid via one or more bone screws 9. The glenosphere 7 may be receivable in an articular member 30 or cup having a concave articular surface 32. These additional example humeral implant systems will be discussed below in more detail.

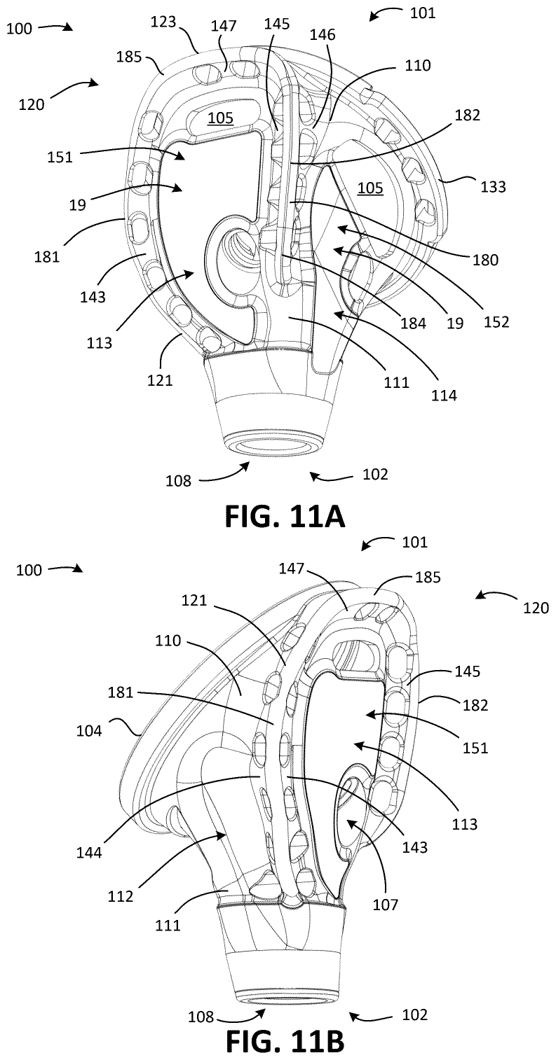

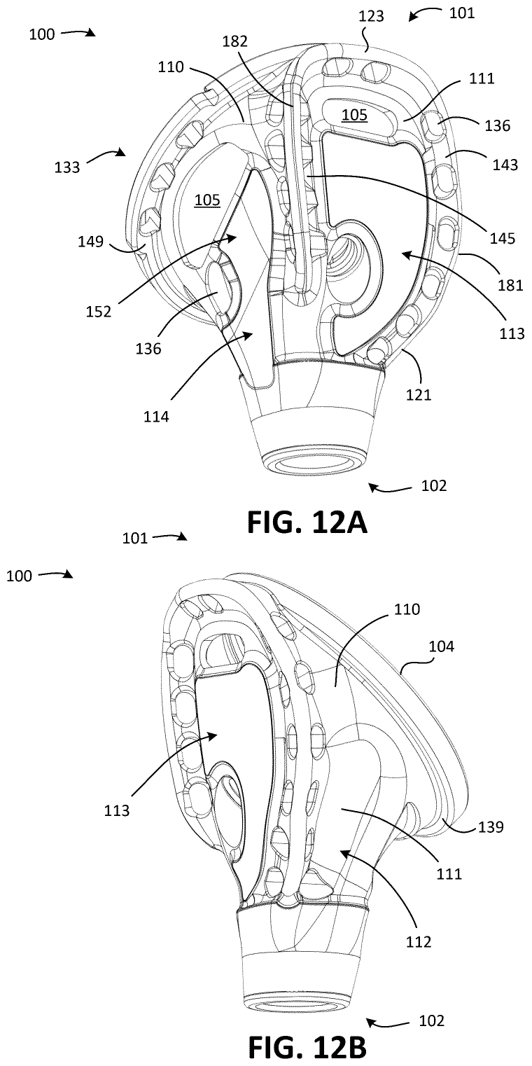

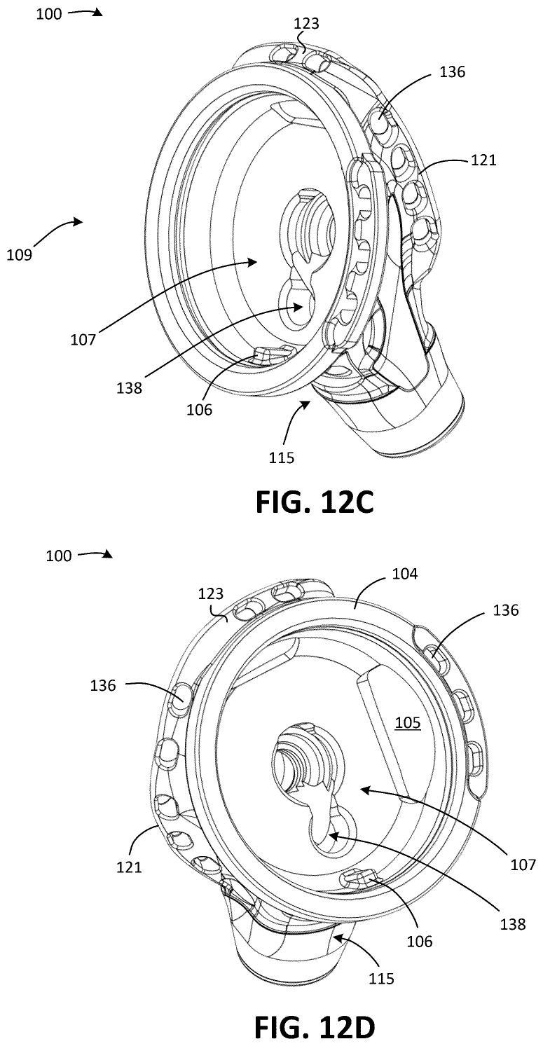

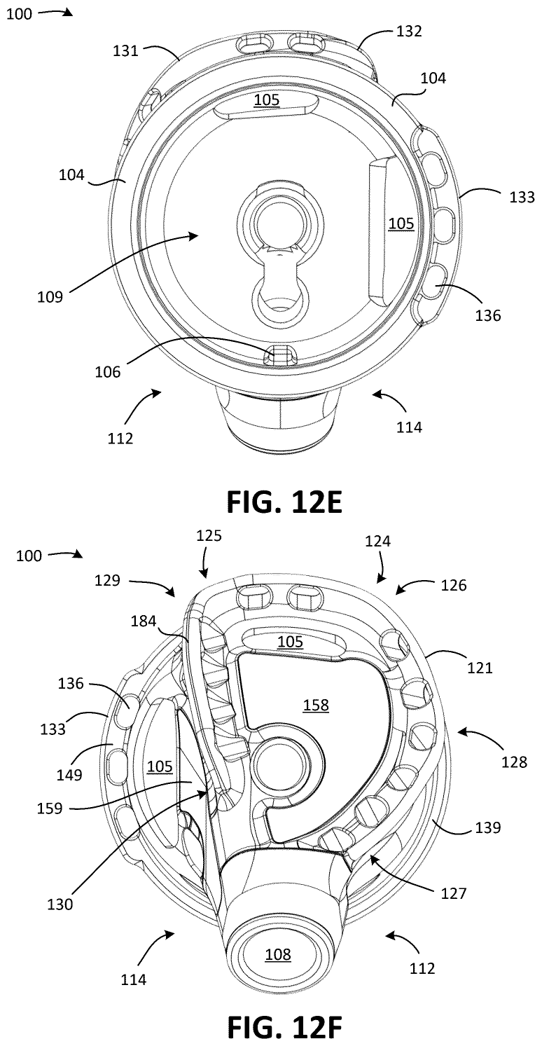

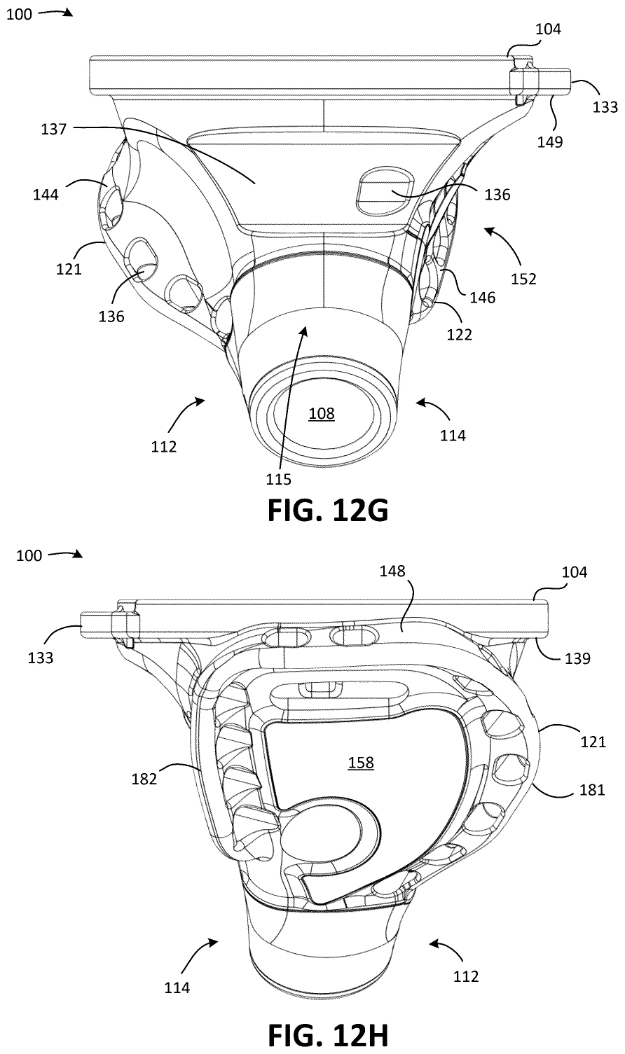

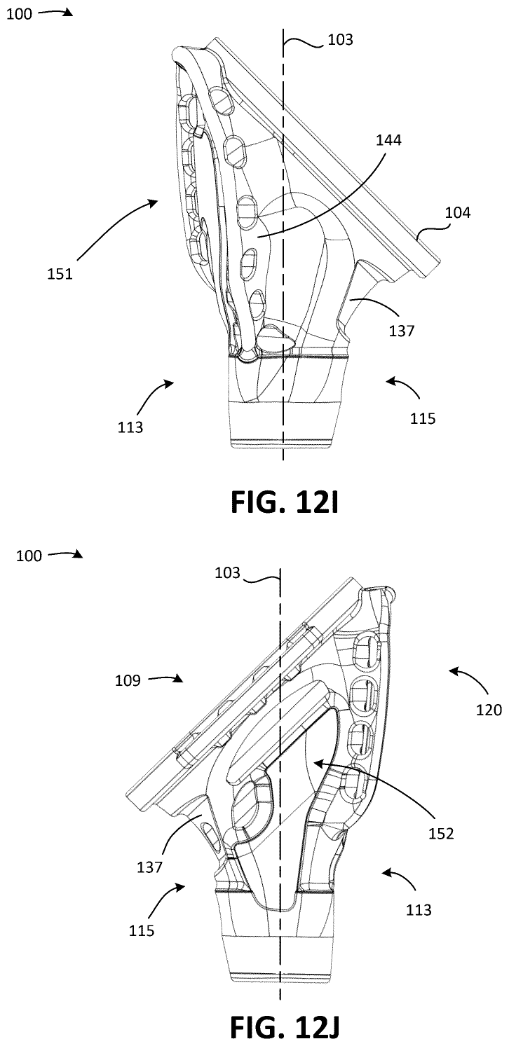

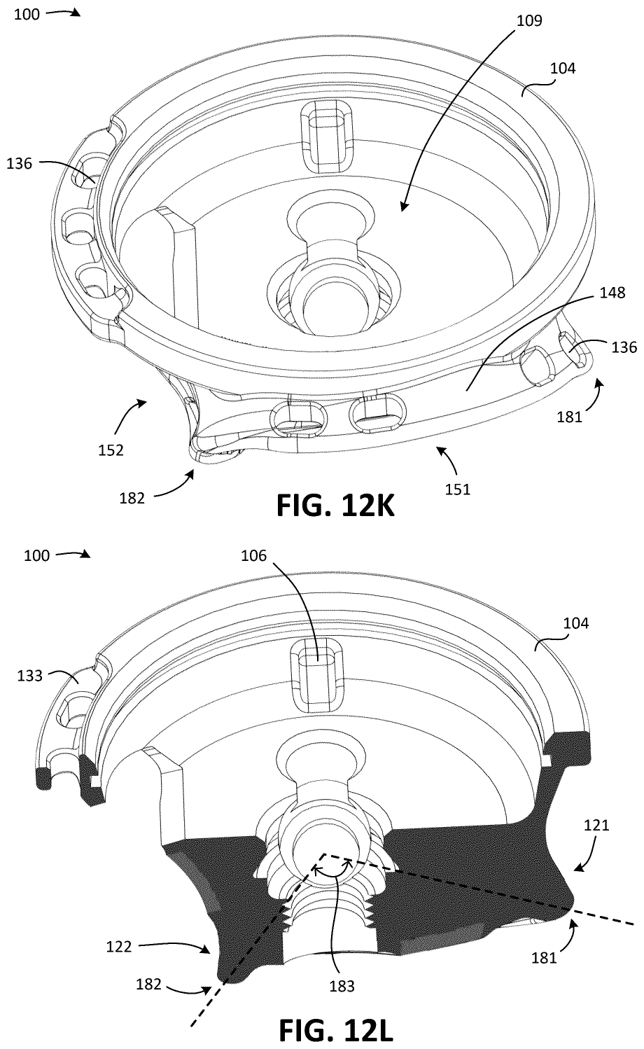

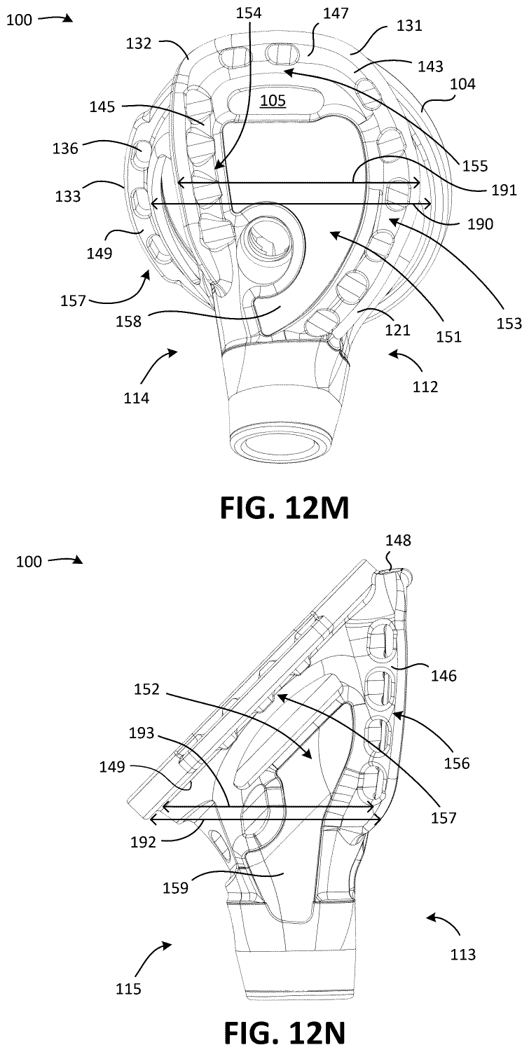

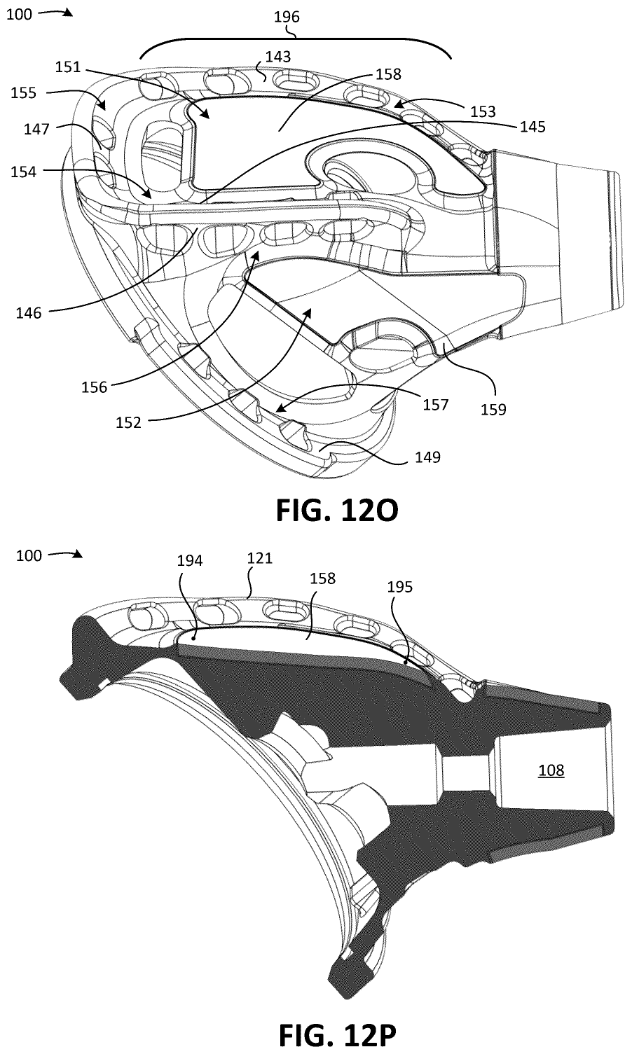

FIGS. 11A-12P illustrate various views of the humeral bone implant or bone implant 100, according to an embodiment of the present disclosure. Specifically, FIGS. 11A and 11B show views of the bone implant 100 shaped for a right humerus and FIGS. 12A-12P show various views of the bone implant 100 shaped for a left humerus.

In general, the bone implant 100 may include a longitudinal axis 103, a rim component 104 having a flange surface 139 at a proximal end 101 of the bone implant 100, a stem attachment feature 108 at a distal end 102 of the bone implant 100, and a body portion 110 extending intermediate the rim component 104 and the stem attachment feature 108.

In some embodiments, the flange surface 139 may serve as a mechanical stop to prevent over insertion of the bone implant 100 into a bone.

In some embodiments, the flange surface 139 may be continuously formed about the rim component 104.

In some embodiments, the flange surface 139 may comprise one or more discontinuous sections. For example, in some embodiments the flange surface 139 and/or the rim component 104 may comprise one or more discrete portions, such as an anterior rim portion 133.

In some embodiments, various components of the bone implant 100 may include one or more suture holes 136 formed through the various components of the bone implant 100 and configured to receive one or more flexible elements 4 therethrough, such as sutures, suture tape 90, etc.

In some embodiments, the one or more suture holes 136 may include cross-sectional shapes that may be round, oval, ellipse, racetrack-shaped, etc., or any combinations thereof.

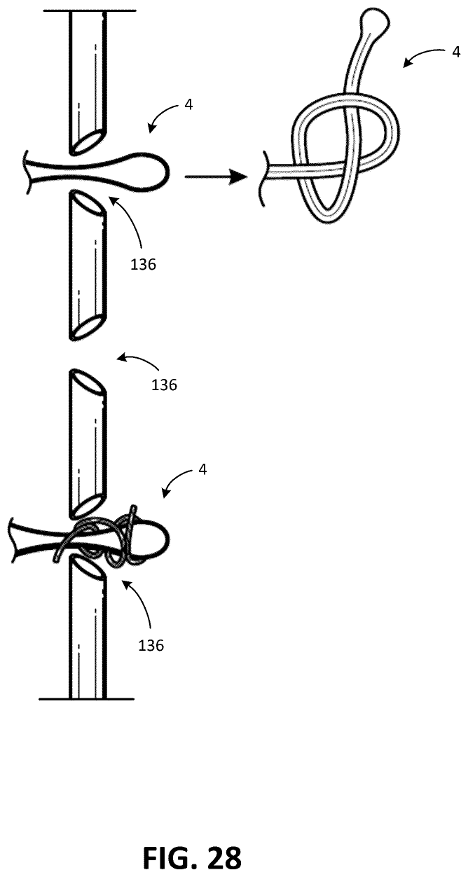

In some embodiments, the one or more suture holes 136 may include cross-sectional shapes that may taper in cross-sectional area from one end of the suture hole to the other end of the suture hole. In these embodiments, the one or more suture holes 136 may include “funnel-like” or other tapered shapes (e.g., see FIG. 28). Such tapered shapes may act as mechanical stops for suture knots (or other features) that are tied on the larger side of the suture hole to trap the suture knot on the larger side of the suture hole and prevent the suture from pulling back through the smaller side of the suture hole.

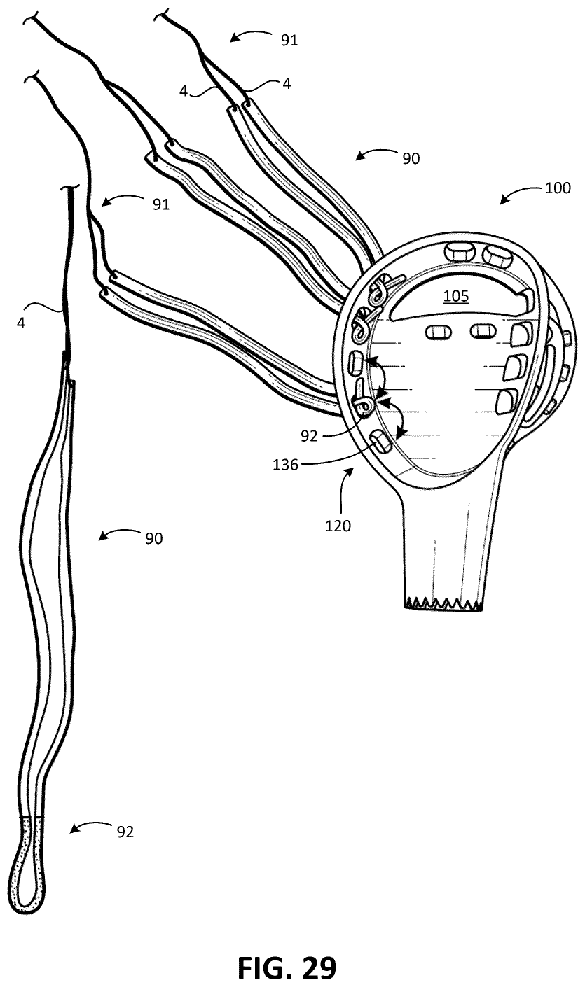

In some embodiments, the one or more flexible elements 4, sutures, or suture tape 90, may be secured to the one or more suture holes 136 via a self-locking suture anchor mechanism, such as a soft suture anchor 92 (e.g., see FIG. 29). Such a mechanism may create a bolus of suture that is too large to pass through the suture holes. This can be done to avoid the need to tie suture knots. In these embodiments, the soft suture anchor 92 may be inserted through a suture hole formed in the bone implant 100 in a first non-expanded state. Once the soft suture anchor 92 is on the other side of the suture hole, the soft suture anchor 92 may then be configured to expand (e.g., by gently tugging on the suture tape 90 to expand the soft suture anchor 92). In this manner, the suture tape 90 may be secured to a suture hole of the bone implant 100. In some embodiments, the soft suture anchor 92 may be fixed relative to the suture tape 90. In some embodiments, the soft suture anchor 92 may be configured to slide relative to the suture tape 90.

In some embodiments, one or more ends of a flexible element, suture, or suture tape 90 may be coupled to each other via a swedge structure 91 to form a single end. In this manner, complexity is reduced by reducing the amount of flexible element ends that need to be passed through suture holes for the system (e.g., see FIG. 29).

In some embodiments, the body portion 110 of the bone implant 100 may generally include an exterior surface 111 having a posterior-facing surface 112, a lateral-facing surface 113, an anterior-facing surface 114, and a medial-facing surface 115.

In some embodiments, the medial-facing surface 115 of the bone implant 100 may include a grooved surface 137 formed therein and configured to receive one or more flexible elements 4 that may be wrapped around the bone implant 100.

In some embodiments, the bone implant 100 may include a raised ridge structure 120 coupled to and projecting away from the exterior surface 111 of the body portion 110.

In some embodiments, the raised ridge structure 120 may include a posterior ridge portion 121 projecting away from the posterior-facing surface 112 and/or the lateral-facing surface 113 of the body portion 110 between the proximal end 101 and the distal end 102 of the bone implant 100.

In some embodiments, the raised ridge structure 120 may include an anterior ridge portion 122 projecting away from the anterior-facing surface 114 and/or the lateral-facing surface 113 of the body portion 110 between the proximal end 101 and the distal end 102 of the bone implant 100.

In some embodiments, the raised ridge structure 120 may include a superior ridge portion 123 projecting away from the lateral-facing surface 113 of the body portion 110 adjacent the rim component 104 and intermediate the posterior ridge portion 121 and the anterior ridge portion 122.

In some embodiments, the superior ridge portion 123 may be continuously joined with at least one of the posterior ridge portion 121 and the anterior ridge portion 122.

In some embodiments, a posterior end 124 of the superior ridge portion 123 may couple with a proximal end 126 or proximal region of the posterior ridge portion 121 to continuously join the posterior ridge portion 121 with the superior ridge portion 123.

In some embodiments, an anterior end 125 of the superior ridge portion 123 may couple with a proximal end 129 or proximal region of the anterior ridge portion 122 to continuously join the anterior ridge portion 122 with the superior ridge portion 123.

In some embodiments, the raised ridge structure 120 may include a first curved portion 131 intermediate the posterior ridge portion 121 and the superior ridge portion 123.

In some embodiments, the raised ridge structure 120 may include a second curved portion 132 intermediate the superior ridge portion 123 and the anterior ridge portion 122.

In some embodiments, the first curved portion 131 may couple intermediate the anterior end 125 of the superior ridge portion 123 and the proximal end 126 of the posterior ridge portion 121 to continuously join the superior ridge portion 123 with the posterior ridge portion 121.

In some embodiments, the second curved portion 132 may couple intermediate the anterior end 125 of the superior ridge portion 123 and the proximal end 129 of the anterior ridge portion 122 to continuously join the superior ridge portion 123 with the anterior ridge portion 122.

In some embodiments, the raised ridge structure 120 may comprise a C-shape when viewed from a lateral direction.

In some embodiments, the posterior ridge portion 121 may include the proximal end 126 or proximal region, the distal end 127 or distal region, and a middle region 128 intermediate the proximal region and the distal region. The proximal region of the posterior ridge portion 121 may curve toward the proximal end 101 of the body portion 110 along a proximal direction, the distal region of the posterior ridge portion 121 may curve toward the distal end 102 of the body portion 110 along a distal direction, and the middle region 128 of the posterior ridge portion 121 may project away from the body portion 110 posteriorly relative to the proximal region and the distal region of the posterior ridge portion 21.

In some embodiments, a distal end 130 of the anterior ridge portion 122 may curve toward the distal end 102 of the body portion 110 along a distal direction.

In some embodiments, the raised ridge structure 120 may include a ridge top 180 comprising a rounded convex surface 185 that defines an apex line 184 extending along the ridge top 180. However, it will also be understood that ridge top 180 may comprise a flat surface, a convex surface, and/or combinations thereof in some embodiments.

In some embodiments, the apex line 184 may be a continuous apex line that extends along the ridge top 180. For example, when one or more of the posterior ridge portion 121, the anterior ridge portion 122, the superior ridge portion 123, the first curved portion 131, and/or the second curved portion 132 are continuously joined together in any combination, the apex line 184 may also be continuous along the ridge top 180 in these continuously joined portions of the raised ridge structure 120. FIGS. 11A-12P illustrate a bone implant 100 embodiment having a continuous apex line that extends along the ridge top 180.

In some embodiments, the apex line 184 may be a discrete apex line that extends along at least one discrete ridge top portion. For example, when one or more of the posterior ridge portion 121, the anterior ridge portion 122, the superior ridge portion 123, the first curved portion 131, and/or the second curved portion 132 are not continuously joined together in any combination, the apex line 184 may also be discontinuous between such disjointed portions of the raised ridge structure 120.

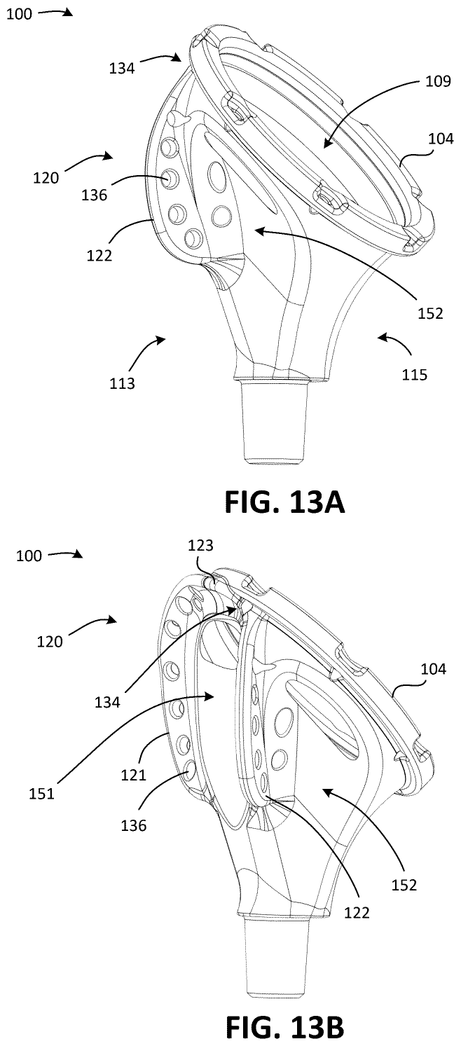

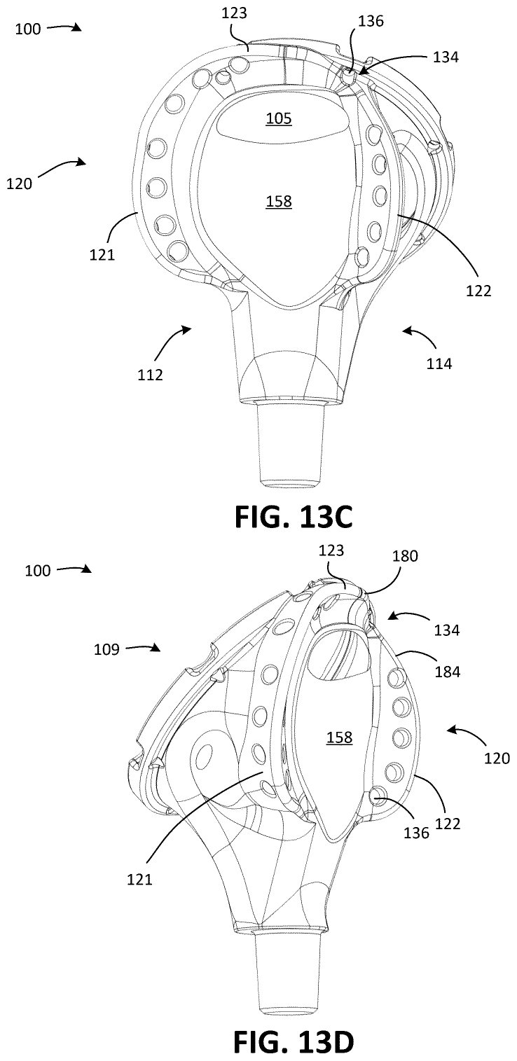

FIGS. 13A-13D illustrate a bone implant 100 according to an alternate embodiment having a raised ridge structure 120 that includes a ridge structure depression 134 intermediate the superior ridge portion 123 and the anterior ridge portion 122. In this embodiment, the ridge top 180 still has some height in the area of the ridge structure depression 134, such that the apex line 184 would still form a continuous apex line in the area of the ridge structure depression 134 (even though the apex line 184 has a lower height in the area of the ridge structure depression 134). However, as can be seen in FIGS. 13A-13D, a suture hole also exists in the area of the ridge structure depression 134. This results in a discrete apex line that is discontinuous between the anterior ridge portion 122 and the superior ridge portion 123.

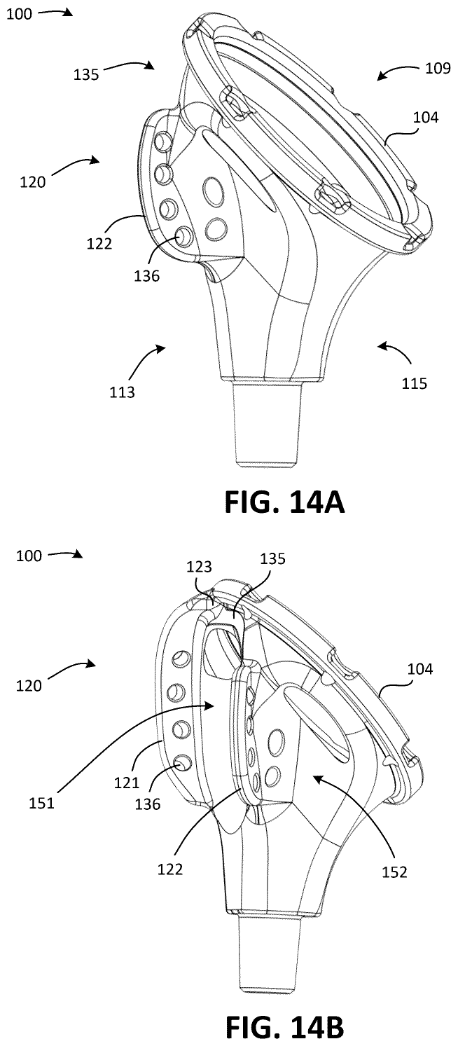

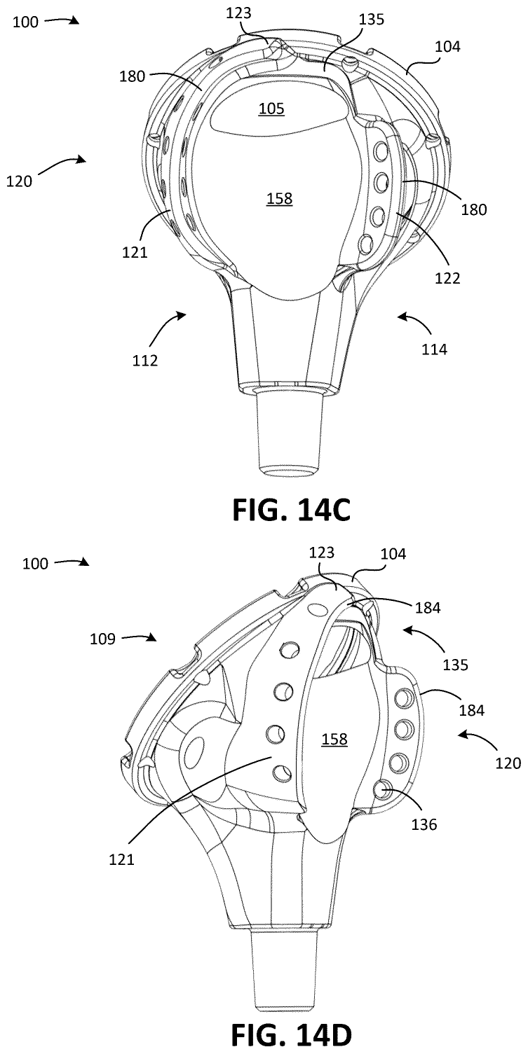

FIGS. 14A-14D illustrate a bone implant 100 according to an alternate embodiment having a raised ridge structure 120 that includes a ridge structure discontinuity 135 intermediate the superior ridge portion 123 and the anterior ridge portion 122 where the ridge top 180 completely disappears. This results in a discrete apex line that is discontinuous between the anterior ridge portion 122 and the superior ridge portion 123 in the area of the ridge structure discontinuity 135.

In some embodiments, the apex line 184 of the ridge top 180 may comprise a horseshoe-shape, a curved arc shape, a C-shape, a bow shape, and an arch shape when viewed from a lateral direction.

In some embodiments,

Referring to FIGS. 12K and 12L, a top perspective view and a corresponding cross-sectional view of the bone implant 100 are shown.

In some embodiments, the apex line 184 along the ridge top 180 may comprise a first apex 181 or first apex line of the posterior ridge portion 121 that projects away from the longitudinal axis 103 of the body portion 110 along a posterior direction, as well as a second apex 182 or second apex line of the anterior ridge portion 122 that projects away from the longitudinal axis 103 of the body portion 110 along an antero-lateral direction (see FIG. 12L).

An angle 183 may be formed between the first apex 181 and the second apex 182 relative to the longitudinal axis 103 of the bone implant 100, as shown in FIG. 12L.

In some embodiments, the angle 183 may be greater than ninety degrees and less than one hundred and forty five degrees.

In some embodiments, the angle 183 may be greater than one hundred degrees and less than one hundred and thirty degrees.

In some embodiments, the angle 183 may be greater than one hundred and five degrees and less than one hundred and twenty degrees.

In some embodiments, the angle 183 may be about one hundred and ten degrees.

In some embodiments, the bone implant 100 may include a first bone fragment basin 151 configured to receive a first bone fragment 1 therein.

In some embodiments, the first bone fragment 1 may comprise a greater tuberosity bone fragment of a humeral bone 3.

In some embodiments, the first bone fragment basin 151 may be defined by the lateral-facing surface 113 that is enclosed between the posterior ridge portion 121 and the anterior ridge portion 122.

In some embodiments, the bone implant 100 may also include a second bone fragment basin 152 configured to receive a second bone fragment 2 therein.

In some embodiments, the second bone fragment 2 may comprise a lesser tuberosity bone fragment of a humeral bone 3.

In some embodiments, the second bone fragment basin 152 may be defined by the anterior-facing surface 114 that is enclosed between the anterior ridge portion 122 and the rim component 104 or anterior rim portion 133 of the bone implant 100.

In some embodiments, a first surface 143 of the posterior ridge portion 121 may define a first side 153 of the first bone fragment basin 151 that may be shaped to abut a first surface 161 of the first bone fragment 1 and prevent posterior rotational movement of the first bone fragment 1 relative to the bone implant 100.

In some embodiments, a first surface 145 of the anterior ridge portion 122 may define a second side 154 of the first bone fragment basin 151 that may be shaped to abut a second surface 162 of the first bone fragment 1 and prevent anterior rotational movement of the first bone fragment 1 relative to the bone implant 100.

In some embodiments, a second surface 146 of the anterior ridge portion 122 may define a first side 156 of the second bone fragment basin 152 that may be shaped to abut a first surface 171 of the second bone fragment 2 and prevent posterior rotational movement of the second bone fragment 2 relative to the bone implant 100.

In some embodiments, a first surface 149 of the rim component 104, or the anterior rim portion 133, may define a second side 157 of the second bone fragment basin 152 that may be shaped to abut a second surface 172 of the second bone fragment 2 and prevent anterior rotational movement of the second bone fragment 2 relative to the bone implant 100. However, it will also be understood that, in other embodiments, a surface projecting from the body portion 110 may also serve to define the second side 157 of the second bone fragment basin 152 instead of the first surface 149 of rim component 104 or anterior rim portion 133.

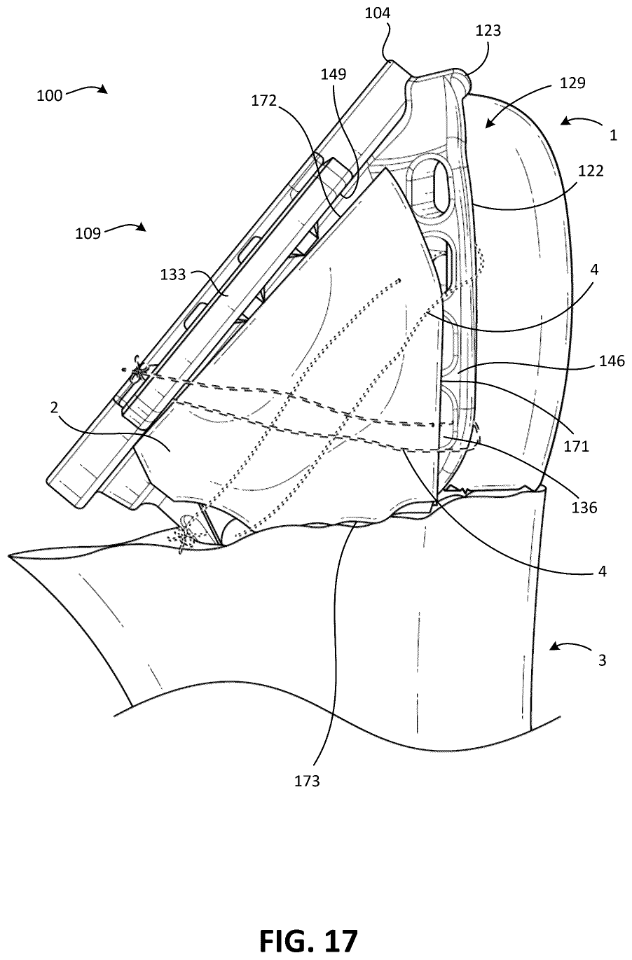

In some embodiments, the first surface 149 of the rim component 104 may be angled toward the proximal end 129 of the anterior ridge portion 122, as shown in FIG. 17.

In some embodiments, a first surface 147 of the superior ridge portion 123 may define a third side 155 of the first bone fragment basin 151 that may be shaped to abut a third surface 163 of the first bone fragment 1 and prevent superior rotational movement of the first bone fragment 1 relative to the bone implant 100.

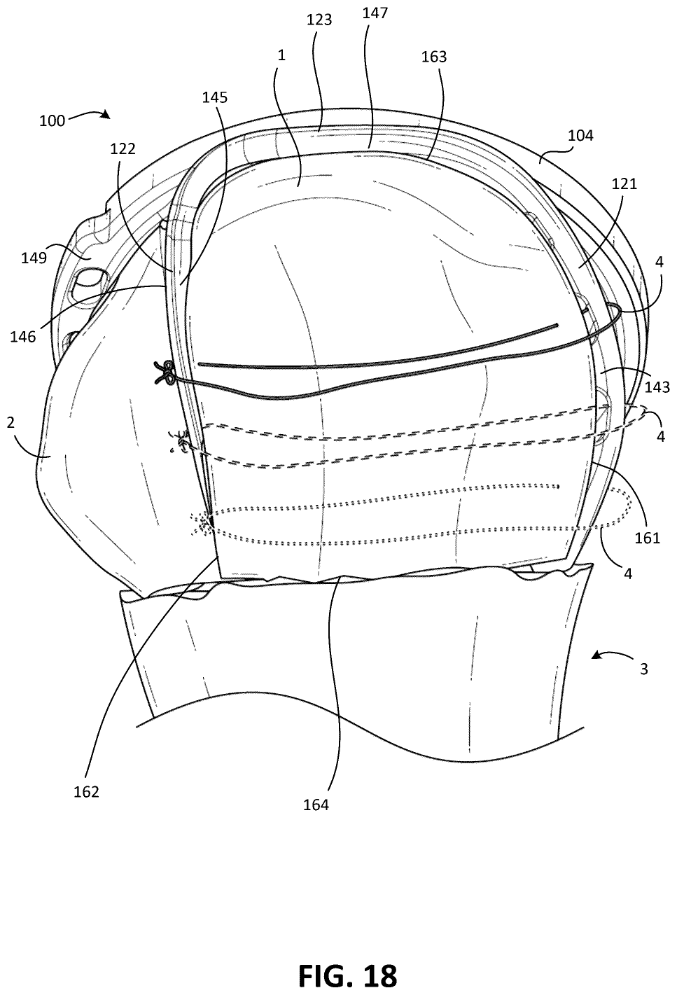

In some embodiments, a fourth surface 164 of the first bone fragment 1 and/or a third surface 173 of the second bone fragment 2 may also abut a surface of the humeral bone 3, as shown in FIGS. 17 and 18.

In some embodiments, the posterior ridge portion 121 comprises a second surface 144 and/or the superior ridge portion 123 comprises a second surface 148.

In some embodiments, at least one of: the first surface 143 of the posterior ridge portion 121, the second surface 144 of the posterior ridge portion 121, the first surface 145 of the anterior ridge portion 122, the second surface 146 of the anterior ridge portion 122, the first surface 147 of the superior ridge portion 123, and the second surface 148 of the superior ridge portion 123 comprises a concave surface.

In some embodiments, at least one of: the first surface 143 of the posterior ridge portion 121, the second surface 144 of the posterior ridge portion 121, the first surface 145 of the anterior ridge portion 122, the second surface 146 of the anterior ridge portion 122, the first surface 147 of the superior ridge portion 123, and the second surface 148 of the superior ridge portion 123 comprises a flat surface.

In some embodiments, at least one of: the first surface 143 of the posterior ridge portion 121, the second surface 144 of the posterior ridge portion 121, the first surface 145 of the anterior ridge portion 122, the second surface 146 of the anterior ridge portion 122, the first surface 147 of the superior ridge portion 123, and the second surface 148 of the superior ridge portion 123 comprises a convex surface.

In some embodiments, the first bone fragment basin 151 may comprise a first plain or first plain surface 158, and the second bone fragment basin 152 may comprise a second plain or second plain surface 159.

In some embodiments, at least one of the first plain surface 158 and the second plain surface 159 may include at least one of: a porous coating 19, rough surface, grit blasting, sintered beads, titanium/calcium phosphate porous coatings, additively manufactured porous structures, biofilms, etc., to help promote bone in-growth and osseointegration of the first and second bone fragments and/or to provide an uneven/irregular surface that may provide increased friction between the bones and the bone implant interface to help prevent movement of the bony structures after they have been secured into position.

Referring to FIG. 12M, in some embodiments the body portion 110 may include a maximum lateral transverse body width 190, the raised ridge structure 120 may include a maximum transverse first basin width 191 intermediate the posterior ridge portion 121 and the anterior ridge portion 122, and the maximum transverse first basin width 191 may be greater than 50% of the maximum lateral transverse body width 190.

In some embodiments, the maximum transverse first basin width 191 may be less than 50% of the maximum lateral transverse body width 190.

In some embodiments, the maximum transverse first basin width 191 may be greater than 70% of the maximum lateral transverse body width 190.

In some embodiments, the maximum transverse first basin width 191 may be greater than 90% of the maximum lateral transverse body width 190.

In some embodiments, the maximum transverse first basin width 191 may equal to the maximum lateral transverse body width 190.

In some embodiments, the maximum transverse first basin width 191 may be greater than the maximum lateral transverse body width 190.

Referring to FIG. 12N, the body portion 110 may include a maximum anterior transverse body width 193 and a maximum transverse first basin width 191.

In some embodiments, the maximum anterior transverse body width 193 may be greater than 50% of the maximum transverse second basin width 192.

In some embodiments, the maximum anterior transverse body width 193 may be less than 50% of the maximum transverse second basin width 192.

In some embodiments, the maximum anterior transverse body width 193 may be greater than 70% of the maximum transverse second basin width 192.

In some embodiments, the maximum anterior transverse body width 193 may be greater than 90% of the maximum transverse second basin width 192.

In some embodiments, the maximum anterior transverse body width 193 may be equal to the maximum transverse second basin width 192.

In some embodiments, the maximum anterior transverse body width 193 may be greater than the maximum transverse second basin width 192.

Referring to FIGS. 120 and 12P, an antero-lateral perspective view and a corresponding cross-sectional view of the bone implant 100 are shown.

In some embodiments, the first plain surface 158 and/or the second plain surface 159 may be substantially flat. For example, in some embodiments the first plain surface 158 may be bounded between a lateral plane (not shown) and a medial plane (not shown) that may be placed adjacent the first plain surface 158, such that: the lateral plane and the medial plane may be parallel to each other; at least one maximum surface point 194 on the first plain surface 158 may be contained within the lateral plane; at least one minimum surface point 195 on the first plain surface 158 may be contained within the medial plane; and a departure distance between the lateral plane and the medial plane may be less than about 10% of a maximum length 196 of the first plain surface 158. This concept may also be applied to the second plain surface 159 to demonstrate that the second plain surface 159 can be substantially flat, in at least some embodiments. However, it will also be understood that the first plain surface 158 and/or the second plain surface 159 may also comprise concave and/or convex surfaces that are not flat, in at least some embodiments.

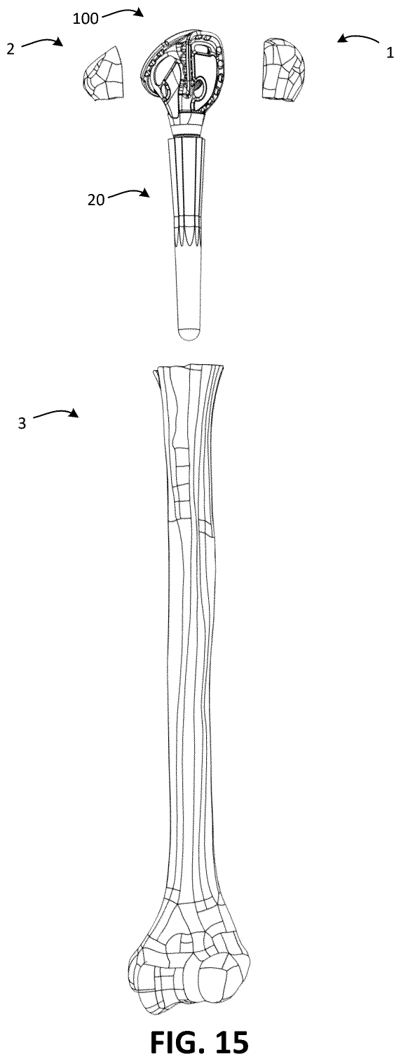

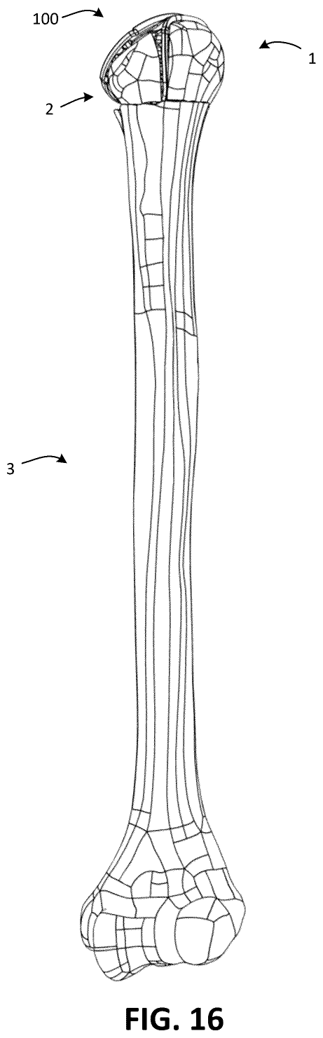

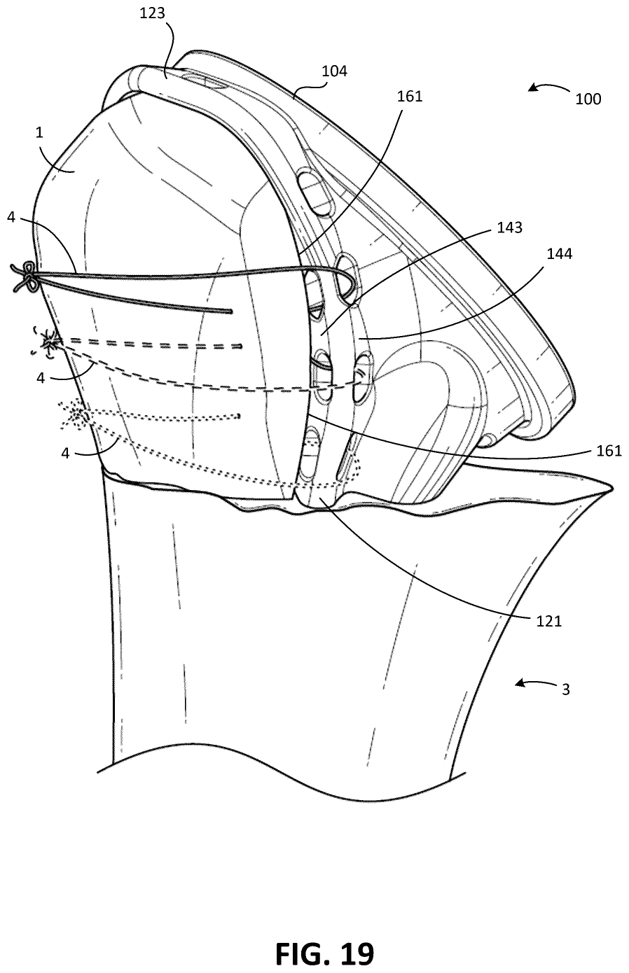

Referring to FIGS. 15-19, a procedure for installing a humeral implant system of the present disclosure is illustrated. Specifically, FIG. 15 shows a left humeral implant system or bone implant 100 relative to a humeral bone 3, a first bone fragment 1 or greater tuberosity, and a second bone fragment 2 or lesser tuberosity, before implantation; FIG. 16 shows an antero-lateral perspective view of the left humeral implant system of FIG. 15 after implantation; FIG. 17 shows a close-up anterior view of the installed implant from FIG. 15 with sutures; FIG. 18 shows a close-up lateral view of the installed implant from FIG. 15 with sutures; and FIG. 19 shows a close-up postero-lateral view of the installed implant from FIG. 15 with sutures.

In a first step of the procedure, the humeral bone 3, the first bone fragment 1, and/or the second bone fragment 2 may each be prepared by performing one or more of the following actions on these bone portions: sectioning, reaming, drilling, rasping, tamping, or otherwise shaping each of these bone portions to prepare them to receive the bone implant 100. For example, an intramedullary canal of the humeral bone 3 may be reamed, drilled, tamped, etc., to prepare the intramedullary canal to receive the stem 20 and/or at least a portion of the distal end 102 of the bone implant 100 therein.

In a second step of the procedure, the stem 20 and/or at least a portion of the distal end 102 of the bone implant 100 may be inserted into the prepared intramedullary canal utilizing an inserter tool (not shown) and a mallet (not shown) to drive the stem 20 and bone implant 100 into the intramedullary canal. Once the stem 20 and bone implant 100 have been properly placed within the intramedullary canal, the first bone fragment 1 and/or the second bone fragment 2 may be provisionally placed into the first bone fragment basin 151 and/or the second bone fragment basin 152, respectively, as shown in FIG. 16.

In a third step of the procedure, the first bone fragment 1 and/or the second bone fragment 2 may be secured to the bone implant 100 by placing one or more flexible elements 4 through the one or more suture holes 136 (and/or or through the bone fragments with a suture passer or suture needle, not shown), and then tying/tensioning the one or more flexible elements 4 to secure the first bone fragment 1 within the first bone fragment basin 151 and the second bone fragment 2 within the second bone fragment basin 152, as shown in FIGS. 17-19.

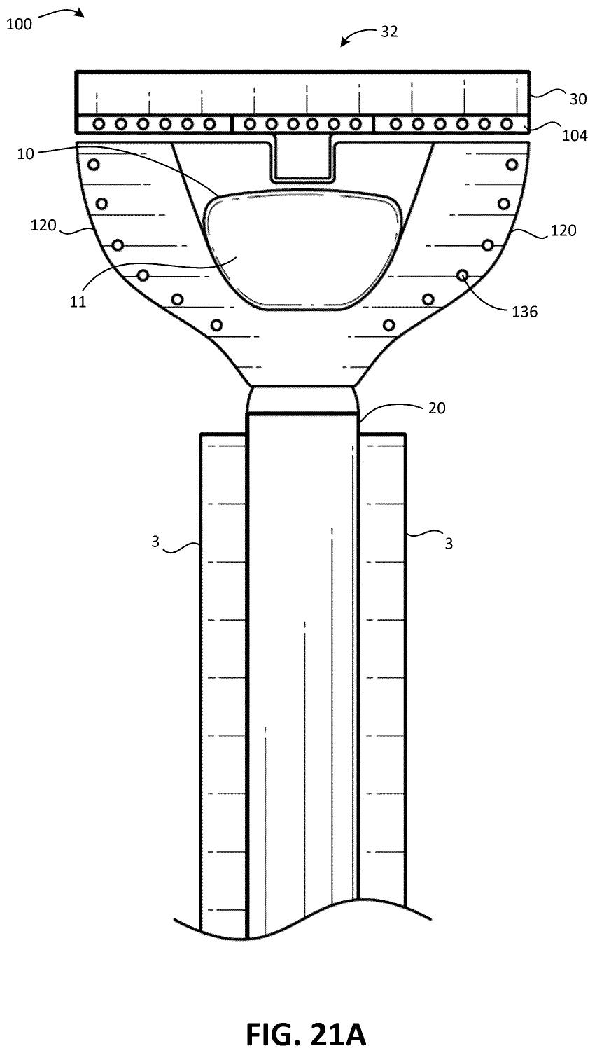

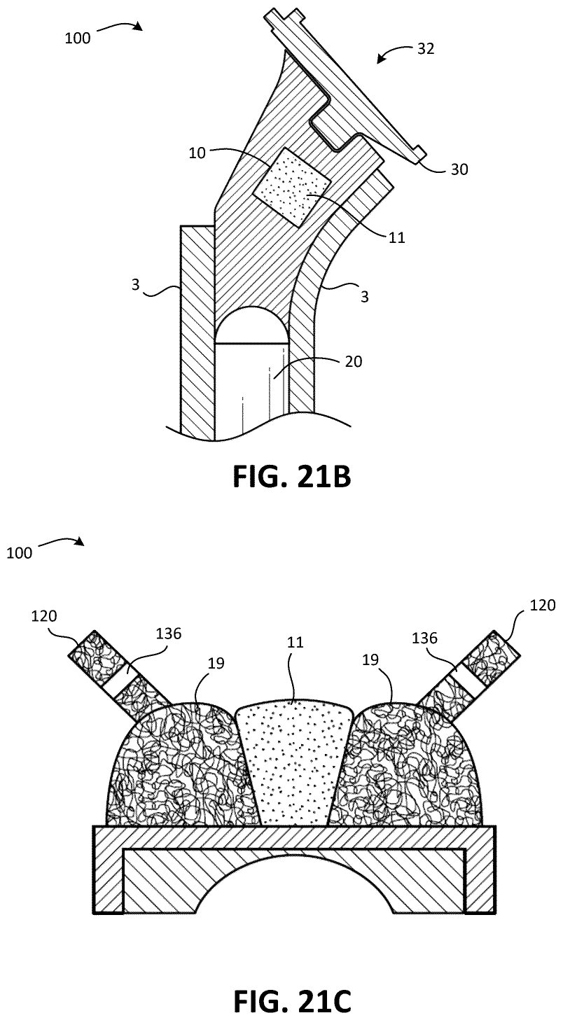

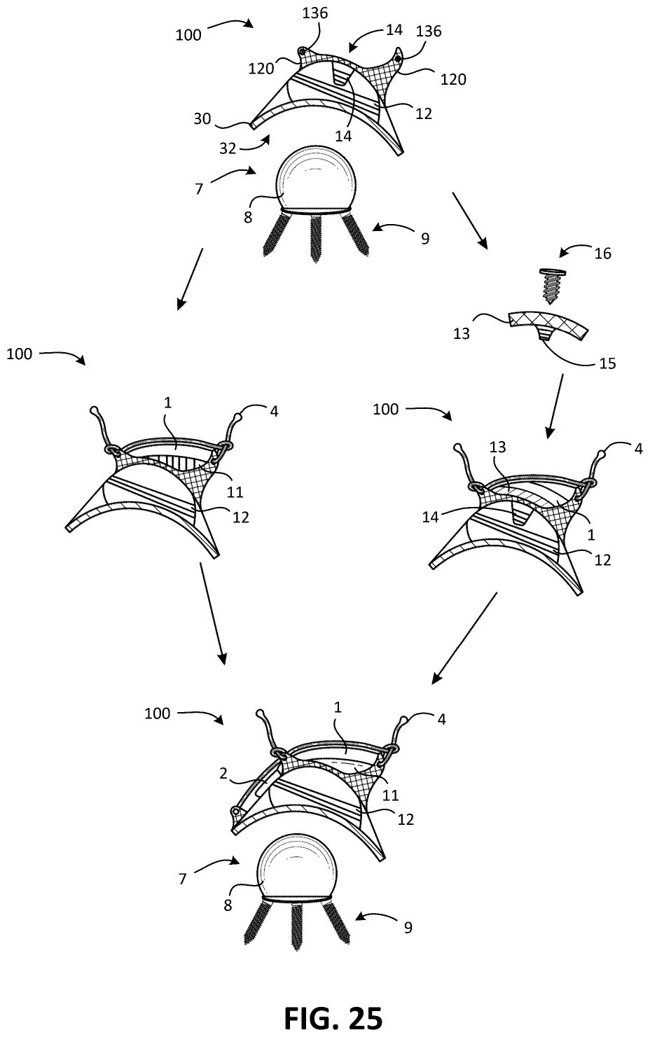

In some embodiments of the procedure, a bone graft 11, or one or more bone grafts, may also be utilized to help provide a secure foundation for the first bone fragment 1 within the first bone fragment basin 151, and/or a secure foundation for the second bone fragment 2 within the second bone fragment basin 152. For example, FIGS. 21A-21C illustrate a bone implant 100 embodiment that includes at least one bone graft cavity 10 configured to receive a bone graft 11 therein. Moreover, FIG. 25 illustrates how a bone graft 11 can be placed beneath the first bone fragment 1 to help build up and/or provide a secure foundation for the first bone fragment 1 (as well as the second bone fragment 2) before it is secured to the bone implant 100 with one or more flexible elements 4.

In some embodiments, a bone grafting system (not shown) may also be utilized with the bone implants of the present disclosure to harvest and shape suitable bone grafts to fit around a bone implant 100 to create one or more bone beds that may facilitate attachment of a bone fragment to a bone implant 100. Some functionality of the bone grafting system may include, but is not limited to: sectioning a bone to create a bone graft with a desired shape, size, curvature, etc.; punching the bone graft with a punch tool (not shown) to create any additional feature, shape, size, curvature, etc., as desired; Drilling or punching one or more holes through the bone graft to receive one or more flexible elements 4 therethrough; preparing at least one surface of the bone graft to facilitate osseointegration (e.g., roughening/rasping a surface of the bone graft to stimulate osseointegration, etc.). It will be understood that the bone grafting system envisioned herein can be adapted to form any desired bone graft shape, size, or feature.

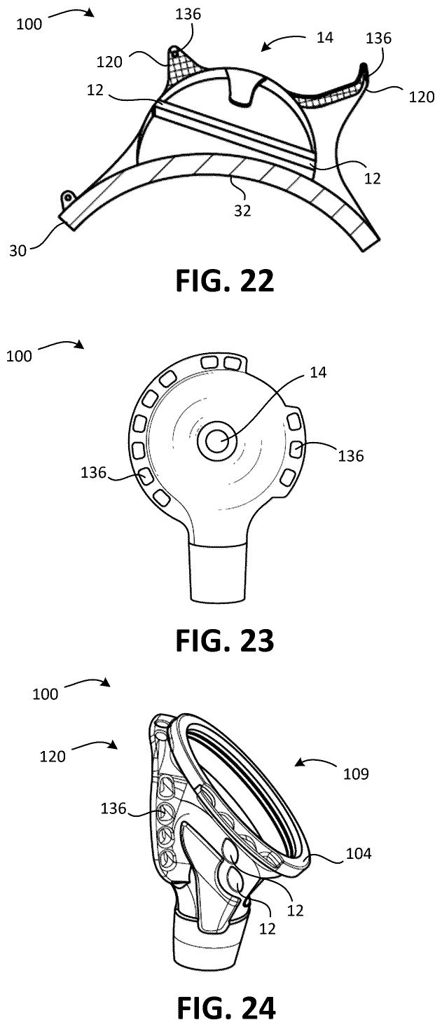

Alternatively, or in addition thereto, in some embodiments of the procedure, an augment, or one or more augments, may also be utilized to help provide a secure foundation for the first bone fragment 1 within the first bone fragment basin 151, and/or a secure foundation for the second bone fragment 2 within the second bone fragment basin 152. For example, FIG. 25 shows an augment 13 with an augment post 15 and an augment fastener 16, as one non-limiting example of an augment structure that may be utilized to help build up and/or provide a secure foundation for the first bone fragment 1 (as well as the second bone fragment 2) before it is secured to the bone implant 100 with one or more flexible elements 4. Moreover, FIG. 25 also shows a bone implant 100 embodiment that includes an augment attachment feature 14 formed therein and configured to receive the augment post 15 and/or the augment fastener 16 to couple the augment 13 to the bone implant 100. Likewise, FIGS. 22 and 24 show bone implants with the augment attachment feature 14 formed therein. It will be understood that the augment 13 can be made of any suitable material(s) including, but not limited to: metals (e.g., titanium, CoCr, stainless steel, any alloy, etc.), ceramics, plastics, PEEK, hydroxyapatite, etc.

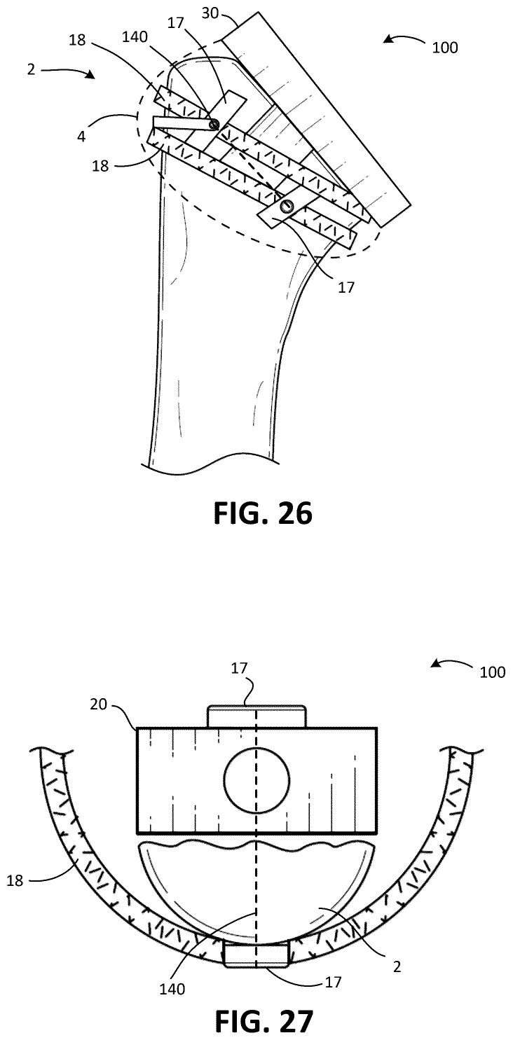

Alternatively, or in addition thereto, in some embodiments of the procedure, a tensioning system may also be utilized to help secure the first bone fragment 1 within the first bone fragment basin 151, and/or secure the second bone fragment 2 within the second bone fragment basin 152. For example, FIGS. 22, 24 and 25 illustrate a bone implant 100 embodiment with one or more tensioning system tunnels 12 formed therethrough and configured to receive a tensioning element 140 therein such as a cable, a cord, a strap, a ligature, etc. In these embodiments, one or more buttons 17 or anchors may also be utilized with the tensioning element 140 to help secure the bone fragment to the bone implant 100, as shown in FIGS. 26 and 27. Two or more anchors or buttons may be placed on opposite sides of the bone implant 100 with the tensioning element 140 connected therebetween to provide a tension force between the two or more anchors or buttons in order to secure the bone fragments to the bone implant 100. In some embodiments, the tensioning system may be locked in position utilizing knots, line locks, any other mechanical construct, etc. Moreover, in some embodiments, cerclage tape 18 and or one or more flexible elements 4 may also be wrapped around the bone fragment/bone implant construct to further secure the bone fragments to the bone implant 100, as shown in FIGS. 26 and 27.

Any procedures or methods disclosed herein comprise one or more steps or actions for performing the described procedure or method. The method steps and/or actions may be interchanged with one another. In other words, unless a specific order of steps or actions is required for proper operation of the embodiment, the order and/or use of specific steps and/or actions may be modified. Moreover, any of the methods or procedures described herein may be further modified by omitting, deleting, and/or adding any of the method, procedure steps, or actions described or contemplated herein.

As defined herein, the term “continuously joined” raised ridge structure portions may include two or more raised ridge structure portions (of any positive, non-zero heights) that are joined together in such a manner that they share at least one common continuous apex line that traverses along the ridge tops of each continuously joined raised ridge structure portion.

As defined herein, the term “substantially continuously joined” raised ridge structure portions comprises raised ridge structure portions that are separated from each other by up to 10 mm of distance with no common continuous apex line traversing intermediate the substantially continuously joined raised ridge structure portions.

It will be understood than any feature or group of features described or contemplated herein with respect to any implant, system, method, or instrument may be combined in any fashion with any other implant, system, method, or instrument that is described or contemplated herein in order to make any number of different implant, system, method, or instrument configurations.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, drawing, or description thereof for the purpose of streamlining the present disclosure. This method of disclosure, however, is not to be interpreted as reflecting an intention that any embodiment or claim require more features than those expressly recited in that embodiment or claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112. It will be apparent to those having skill in the art that changes may be made to the details of the above-described embodiments without departing from the underlying principles set forth herein.

The phrases “connected to”, “coupled to”, and “in communication with” refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, as well as components that are removably and/or non-removably coupled with each other. The term “abutting” refers to items that may be in direct physical contact with each other, although the items may not necessarily be attached together. The phrase “fluid communication” refers to two or more features that are connected such that a fluid within one feature is able to pass into another feature. Moreover, as defined herein the term “substantially” means within +/−10% of a target value, measurement, or desired characteristic.

While specific embodiments and applications of the present disclosure have been illustrated and described, it is to be understood that the scope of this disclosure or the appended claims are not limited to the precise configuration and components disclosed herein. Various modifications, changes, and variations which will be apparent to those skilled in the art may be made in the arrangement, operation, and details of the devices, implants, systems, methods, or instruments disclosed herein.

What is claimed is:

1. A bone implant comprising:
a rim component at a proximal end of the bone implant;
a stem attachment feature at a distal end of the bone implant;
a body portion intermediate the rim component and the stem attachment feature, the body portion comprising:
an exterior surface comprising:
a posterior-facing surface;
a lateral-facing surface; and
an anterior-facing surface; and
a raised ridge structure coupled to and projecting away from the exterior surface of the body portion, the raised ridge structure comprising:
a posterior ridge portion projecting away from the posterior-facing surface of the body portion between the proximal end and the distal end of the bone implant;
an anterior ridge portion projecting away from the anterior-facing surface of the body portion between the proximal end and the distal end of the bone implant; and
a superior ridge portion projecting away from the lateral-facing surface of the body portion adjacent the rim component and intermediate the posterior ridge portion and the anterior ridge portion;
wherein the superior ridge portion is continuously joined with at least one of:
the posterior ridge portion; and
the anterior ridge portion.

2. The bone implant of claim 1, wherein:
a posterior end of the superior ridge portion couples with a proximal end of the posterior ridge portion to continuously join the posterior ridge portion with the superior ridge portion; and
an anterior end of the superior ridge portion couples with a proximal end of the anterior ridge portion to continuously join the anterior ridge portion with the superior ridge portion.

3. The bone implant of claim 2, wherein the raised ridge structure further comprises:
a first curved portion intermediate the posterior ridge portion and the superior ridge portion; and
a second curved portion intermediate the superior ridge portion and the anterior ridge portion;
wherein:
the first curved portion couples intermediate the anterior end of the superior ridge portion and the proximal end of the posterior ridge portion to continuously join the superior ridge portion with the posterior ridge portion; and
the second curved portion couples intermediate the anterior end of the superior ridge portion and the proximal end of the anterior ridge portion to continuously join the superior ridge portion with the anterior ridge portion.

4. The bone implant of claim 1, wherein the raised ridge structure comprises a C-shape when viewed from a lateral direction.

5. The bone implant of claim 1, wherein the raised ridge structure comprises a ridge top having a rounded convex surface defining an apex line that extends along the ridge top.

6. The bone implant of claim 5, wherein the apex line comprises a continuous apex line that extends along the ridge top.

7. The bone implant of claim 5, wherein the apex line comprises a discrete apex line that extends along the ridge top.

8. A bone implant comprising:
a body portion comprising:
a proximal end;
a distal end; and
an exterior surface extending between the proximal end and the distal end of the body portion, the exterior surface comprising:
a posterior-facing surface;
a lateral-facing surface; and
an anterior-facing surface;
a raised ridge structure coupled to and projecting away from the exterior surface of the body portion, the raised ridge structure comprising:
a posterior ridge portion projecting away from the posterior-facing surface of the body portion between the proximal end and the distal end of the body portion; and
an anterior ridge portion projecting away from the anterior-facing surface of the body portion between the proximal end and the distal end of the bone implant;
a first bone fragment basin configured to receive a first bone fragment therein, the first bone fragment basin defined by the lateral-facing surface enclosed between the posterior ridge portion and the anterior ridge portion; and
a second bone fragment basin configured to receive a second bone fragment therein, the second bone fragment basin defined by the anterior-facing surface enclosed between the anterior ridge portion and a rim component of the bone implant;
wherein:
a first surface of the posterior ridge portion defining a first side of the first bone fragment basin is shaped to abut a first surface of the first bone fragment and prevent posterior rotational movement of the first bone fragment relative to the bone implant;
a first surface of the anterior ridge portion defining a second side of the first bone fragment basin is shaped to abut a second surface of the first bone fragment and prevent anterior rotational movement of the first bone fragment relative to the bone implant;
a second surface of the anterior ridge portion defining a first side of the second bone fragment basin is shaped to abut a first surface of the second bone fragment and prevent posterior rotational movement of the second bone fragment relative to the bone implant; and
a first surface of the rim component defining a second side of the second bone fragment basin is shaped to abut a second surface of the second bone fragment and prevent anterior rotational movement of the second bone fragment relative to the bone implant.

9. The bone implant of claim 8, wherein the raised ridge structure further comprises:
a superior ridge portion projecting away from the lateral-facing surface of the body portion adjacent the rim component and intermediate the posterior ridge portion and the anterior ridge portion.

10. The bone implant of claim 9, wherein:
a first surface of the superior ridge portion defining a third side of the first bone fragment basin is shaped to abut a third surface of the first bone fragment and prevent superior rotational movement of the first bone fragment relative to the bone implant.

11. The bone implant of claim 10, wherein at least one of: the first surface of the posterior ridge portion, the first surface of the anterior ridge portion, and the first surface of the superior ridge portion comprises a concave surface.

12. The bone implant of claim 9, wherein the superior ridge portion is continuously joined with at least one of:
the posterior ridge portion; and
the anterior ridge portion.

13. The bone implant of claim 8, wherein the first surface of the rim component is angled toward a proximal end of the anterior ridge portion.

14. The bone implant of claim 13, wherein the first surface of the rim component is configured to abut the second surface of the second bone fragment to prevent anterior rotational movement and superior rotational movement of the second bone fragment relative to the bone implant.

15. A bone implant comprising:
a body portion comprising:
a longitudinal axis;
a proximal end;
a distal end; and
an exterior surface extending between the proximal end and the distal end of the body portion, the exterior surface comprising:
a posterior-facing surface;
a lateral-facing surface; and
an anterior-facing surface;
a raised ridge structure coupled to and projecting away from the exterior surface of the body portion, the raised ridge structure comprising:
a posterior ridge portion projecting away from the posterior-facing surface of the body portion between the proximal end and the distal end of the body portion; and
an anterior ridge portion projecting away from the anterior-facing surface of the body portion between the proximal end and the distal end of the bone implant;
wherein:
a first apex of the posterior ridge portion projects away from the longitudinal axis of the body portion along a posterior direction;
a second apex of the anterior ridge portion projects away from the longitudinal axis of the body portion along an antero-lateral direction; and
an angle formed between the first apex and the second apex relative to the longitudinal axis of the body portion is greater than ninety degrees and less than one hundred and forty five degrees.

16. The bone implant of claim 15, wherein the posterior ridge portion comprises:
a proximal region;
a distal region; and
a middle region intermediate the proximal region and the distal region;
wherein:
the proximal region of the posterior ridge portion curves toward the proximal end of the body portion along a proximal direction;
the distal region of the posterior ridge portion curves toward the distal end of the body portion along a distal direction; and
the middle region of the posterior ridge portion projects away from the body portion posteriorly relative to the proximal region and the distal region of the posterior ridge portion. The bone implant of claim 15, wherein the posterior ridge portion comprises a C-shape when

17. viewed laterally.

18. The bone implant of claim 15, wherein the raised ridge structure further comprises a superior ridge portion that is continuously joined with at least one of:
the posterior ridge portion; and
the anterior ridge portion.

19. The bone implant of claim 15, wherein:
the body portion comprises a maximum lateral transverse body width;
the raised ridge structure comprises a maximum transverse first basin width intermediate the posterior ridge portion and the anterior ridge portion; and
the maximum transverse first basin width is greater than 50% of the maximum lateral transverse body width.

20. The bone implant of claim 15, wherein:
the body portion comprises a first basin having a first plain surface bounded between a lateral plane and a medial plane;
wherein:
the lateral plane and the medial plane are parallel to each other;
at least one maximum surface point on the first plain surface is contained within the lateral plane;
at least one minimum surface point on the first plain surface is contained within the medial plane; and
a departure distance between the lateral plane and the medial plane is less than 10% of a maximum length of the first plain surface.
